(19) United States
(12) Patent Application Publication
(10) Pub. No.: US 2022/0283153 A1
(43) Pub. Date: Sep. 8, 2022

(54) COMPOSITIONS AND METHODS FOR DETECTING SEPSIS

(71) Applicant: Seattle Children's Hospital d/b/a Seattle Children's Research Institute, Seattle, WA (US)

(72) Inventors: Thomas D. Yager, Seattle, WA (US); Dayle L. Sampson, Seattle, WA (US); Silvia Cermelli, Seattle, WA (US); Therese Seldon, Seattle, WA (US); Krupa Navalkar, Seattle, WA (US); Adrian Piliponsky, Seattle, WA (US)

(73) Assignee: Seattle Children's Hospital d/b/a Seattle Children's Research Institute, Seattle, WA (US)

(21) Appl. No.: 17/632,495

(22) PCT Filed: Aug. 4, 2020
(86) PCT No.: PCT/US20/44850

§ 371 Date: Feb. 2, 2022

Related U.S. Application Data
(60) Provisional application No. 62/882,696, filed on Aug. 5, 2019.

(51) Int. Cl. G01N 33/543; G01N 33/68

(52) U.S. Cl. CPC G01N 33/54313; G01N 33/6893; G01N 2800/52; G01N 2800/26

(57) ABSTRACT

The disclosure provides a panel of biomarkers that individually or in combination can indicate the presence of sepsis as distinguishable from other non-infection related inflammatory conditions. The disclosed biomarkers and related reagents and kits provide strategies for detecting, treating, and monitoring sepsis in subjects. In aspect, the disclosure provides a method for detecting sepsis, comprising contacting a biological sample obtained from the subject with an affinity reagent that specifically binds to one or more of the disclosed novel biomarkers, and detecting differential expression of the one or more biomarkers by detecting binding of the affinity reagent to the biomarker. The method can incorporate use of additional known biomarkers. The method can further comprise treating a subject determined to have sepsis. In some embodiments, the subject is a human subject less than 20 years old.

### CROSS-REFERENCE TO RELATED APPLICATION

This application claims the benefit of U.S. Provisional Application No. 62/882,696, filed Aug. 5, 2019, the entire disclosure of which is incorporated by reference herein in its entirety.

### BACKGROUND

Sepsis is a life-threatening dysregulated host response to infection leading to organ dysfunction, and it is one of the most common causes of childhood death and disability worldwide. Identifying sepsis in pediatric patients is more challenging than in adult patients, as changes in vital signs may not be as severe in children, which can lead to treatment delay. Subsequently, delayed antimicrobial therapy (even hourly delays) in pediatric sepsis is associated with significantly increased mortality. On the other hand, overuse of antibiotics in critically-ill children was shown to be associated with antibiotic-resistant infections, necrotizing enterocolitis, invasive candidiasis, bronchopulmonary dysplasia, and death. These observations highlight the need for measurements that can augment clinical variables to facilitate differentiation between sepsis and infection-negative systemic inflammation (INSI) in children. A four gene transcriptional signature that achieves a high level of discrimination between critically ill children with sepsis and INSI was previously reported. Proteomics is a potentially very informative approach for identifying sepsis biomarker proteins, understanding the pathophysiological mechanisms of the complex sepsis syndrome, detecting and diagnosing sepsis in a subject, effecting decisions relating to sepsis treatment, and monitoring effectiveness of sepsis treatment. Approaches such as two-dimensional polyacrylamide gel electrophoresis and liquid-chromatography mass-spectrometry have been used to successfully identify large-scale changes in proteins found in the serum or plasma of septic patients or animals subjected to sepsis models, such as cecal ligation and puncture. However, these technologies were subsequently found to not exhibit sufficient sensitivity for identifying changes in low-abundance or low molecular weight proteins. Specifically, the typical dynamic range of detection for liquid-chromatography mass-spectrometry is 4-6 orders of magnitude, while in serum a dynamic range of measurements greater than 7 orders of magnitude is needed to identify proteins in septic subjects present at ng/mL concentrations or lower. None of the prior methods have been applied to serum protein changes in pediatric sepsis populations.

Accordingly, despite advances in the art for detecting and characterizing circulating protein marker profiles, there remains a need to sensitive and efficient methods to detect and characterize sepsis in subjects. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated they become better understood by reference to the following detailed description, when taken in conjunction with the accompanying drawings, wherein:

FIGS. 1A and 1B graphically illustrate top up- and down-regulated differentially expressed proteins between the sepsis and post-cardiopulmonary bypass groups. Empirical cumulative density function plots for haptoglobin (FIG. 1A) and (FIG. 1B) hemoglobin are shown for the INSI (solid circles) and sepsis (solid squares) patients. Haptoglobin was the most up-regulated serum protein, while hemoglobin was the most down-regulated serum protein in the sepsis patients.

FIGS. 2A and 2B depict weighted gene correlation network analysis (WGCNA) results. Module-trait relationships between the WGCNA protein modules and the sepsis and morbidity/mortality or post-cardiopulmonary bypass-related clinical traits are shown. The scale bar in FIG. 2A indicates the shading that corresponds to positive and negative Pearson correlation coefficients, respectively, with more intense shading representing Pearson correlation coefficients closer to 1 and −1. This is the correlation between the module eigengene and clinical characteristics. *, **, and *** represent p-values of p<0.05, p<0.01, and p<0.001, respectively.

FIG. 3 is an ingenuity pathway analysis (IPA). The brown WGCNA protein module proteins determined to be significantly differentially expressed in Table 5 were subjected to IPA to identify known direct (solid lines) and indirect (dashed lines) network interactions. Thirty-three brown module proteins, as determined by IPA, and 15 upstream or downstream proteins (user-defined number), which were added with the “grow” feature, were found to be connected via direct or indirect interactions. The added upstream/downstream proteins include calpain, Gm-csf, GPIIB-IIIA, IL1A, IL22, Immunoglobulin, LOXL2, Mapk, N-cor, OCLN, STAT3, STAT5a/b, TF, TJP1, PIAS3, and TYK2 from the IPA database.

FIG. 4 illustrates the principal component analysis of the sepsis and post-cardiopulmonary bypass patient samples following SOMAscan® analysis. The clinically overt sepsis (SEPSIS, upper grouping) and post-cardiopulmonary bypass (INSI, lower grouping) patient serum samples formed two distinct groups following principal component analysis. Sample SEP-009 was identified as a statistical outlier by this analysis.

FIG. 5 is a consort diagram and depicts how the subjects were included for differential expression and secondary analysis following SOMAscan® analysis. CBP: cardiopulmonary bypass; INSI: post-cardiopulmonary bypass; SEP: sepsis; VIR: viral infection.

FIGS. 6A-6H is a set of examples of up- and down-regulated differentially expressed proteins between the sepsis and post-cardiopulmonary bypass groups. Empirical cumulative density function plots for (FIG. 6A) haptoglobin, (FIG. 6B) serum amyloid A-1, (FIG. 6C) IL1RL1, (FIG. 6D) lactadherin, (FIG. 6E) hemoglobin, (FIG. 6F) ankyrin-2, (FIG. 6G) troponin-1, cardiac muscle, and (FIG. 6H) pleiotrophin are shown for the INSI (solid circles) and sepsis (solid squares) patients. Proteins in panels (FIGS. 6A-6D) were up-regulated while proteins in panels (FIGS. 6E-6H) were down-regulated in the sepsis patients.

FIG. 7 is a cluster dendrogram from the weighted gene correlation network analysis (WGCNA). Every vertical line corresponds to a SOMAmer. Hierarchical clustering of the branches is based on grouping of highly correlated SOMAmers. SOMAmers in the “gray” module are those which did not belong to any of the remaining seven modules. No dynamic tree-cutting algorithm was applied. The most significant clinical traits for each module were identified by binning with respect to p-value (high: p≤0.001; moderate: 0.001<p≤0.01; low: 0.01<p≤0.05).

FIG. 8 demonstrates total weighted gene correlation network analysis module trait relationships. Each column corresponds to a module eigengene, each row corresponds to a trait. Each cell contains the corresponding correlation and p-value. The scale bar in FIG. 8 indicates the shading that corresponds to positive and negative Pearson correlation coefficients, respectively, with more intense shading representing Pearson correlation coefficients closer to 1 and −1. p-values are coded through asterisks, p-value<0.001: ***, <0.01: **, <0.05: *.

FIG. 9 is a module eigengene correlation heatmap for WGCNA modules. Clustering of the protein modules in relation to the SeptiSCORE parameter was evaluated. The “brown” WGCNA module most closely aligned with the SeptiSCORE parameter in the heatmap.

### DETAILED DESCRIPTION

This disclosure is based on a retrospective cohort study of subjects to identify reliable markers for sepsis. As indicated above, sepsis is a life-threatening organ dysfunction caused by a dysregulated host response to infection and is a leading cause of death and disability among children worldwide. Identifying sepsis in pediatric patients is difficult and can lead to treatment delay.

As described in more detail below, to assess the host proteomic response to infection an aptamer-based multiplexed proteomics approach was used to identify novel serum protein changes that might help distinguish between pediatric sepsis and infection-negative systemic inflammation and, hence, improve sensitivity and specificity of the diagnosis of sepsis over current clinical criteria approaches Briefly, the study involved a retrospective, observational cohort study of subjects in pediatric and cardiac intensive care units at Seattle Children's Hospital in Seattle, Wash. The subjects included 40 children with clinically overt sepsis and 30 children immediately following cardiopulmonary bypass surgery (infection-negative systemic inflammation (INSI) control subjects). Children with sepsis had a confirmed or suspected infection, two or more systemic inflammatory response syndrome criteria, and cardiovascular and/or pulmonary organ dysfunction.

Serum samples from 35 of the sepsis and 28 of the bypass surgery subjects were screened with an aptamer-based proteomic (slow off-rate modified aptamer panel or SOMAmer®) platform, which measured 1,305 proteins in search of large-scale serum protein expression pattern changes in sepsis. Novel proteins, as well as previously described proteins, highly differentially expressed between children with sepsis and children with INSI were identified. A total of 111 proteins were significantly differentially expressed between the sepsis and control groups, using the LIMMA (linear modeling) and Boruta (decision trees) R packages, of which 55 proteins were previously identified in sepsis patients. Weighted gene correlation network analysis identified 76 proteins that correlated highly with clinical sepsis traits, 27 of which had not been previously reported for sepsis.

The serum protein changes identified with the aptamer-based multiplexed proteomics can distinguish between sepsis and non-infectious systemic inflammation. It has utility for detecting and diagnosing sepsis, leading to improved treatment for sepsis as well as monitoring of sepsis treatment. As a result, this disclosure is applicable to improving detection sensitivity and specificity in the diagnosis of sepsis over current clinical approaches, as well as monitoring sepsis in subjects, treating sepsis, and monitoring effectiveness of sepsis treatment.

In accordance with the foregoing, the disclosure provides a method for detecting sepsis in a subject. The method comprises contacting a biological sample obtained from the subject with an affinity reagent that specifically binds to a biomarker, wherein the biomarker is selected from the biomarkers disclosed in Table 1. The method also comprises detecting differential expression of the biomarker by detecting binding of the affinity reagent to the biomarker. A determined differential expression of the biomarker indicates sepsis in the subject.

Table 1 is a table of novel differentiated expressed proteins, as designated by the binding SOMAmer, Entrez gene symbol, and protein name. The degree of differential expression is indicated. INSI: Infection-negative systemic inflammation; LIMMA: Linear models for microarray data

| | Entrez | | | Log2 fold |
|---|---|---|---|---|
| | Gene | Protein Name (SOMAmer | Adjusted P- | (Sepsis vs. |
| SOMAmer | Symbol | target) | Value* | INSI) |
| SL014896 | ANK2 | Ankyrin-2 | 6.84E−47 | −5.89 |
| SL003770 | SFRP1 | Secreted frizzled-related | 1.16E−15 | −3.94 |
| | | protein 1 | | |
| SL008381 | CTSF | Cathepsin F | 5.91E−14 | −0.93 |
| SL008023 | HAPLN1 | Hyaluronan and | 3.94E−13 | −1.61 |
| | | proteoglycan link protein 1 | | |
| SL004652 | WIF1 | Wnt inhibitory factor 1 | 1.09E−12 | −0.85 |
| SL010328 | MED1 | Mediator of RNA | 1.28E−12 | −1.21 |
| | | polymerase II transcription | | |
| | | subunit 1 | | |
| SL004336 | FGF18 | Fibroblast growth factor 18 | 1.51E−12 | −1.63 |
| SL002704 | PTN | Pleiotrophin | 1.77E−12 | −3.95 |
| SL003994 | BMP1 | Bone morphogenetic | 3.32E−12 | −0.94 |
| | | protein 1 | | |
| SL008102 | MDH1 | Malate dehydrogenase, | 6.73E−12 | −1.65 |
| | | cytoplasmic | | |
| SL019096 | PIAS4 | E3 SUMO-protein ligase | 1.50E−11 | 0.88 |
| | | PIAS4 | | |
| SL004858 | GFRA1 | GDNF family receptor | 1.90E−10 | −1.55 |
| | | alpha-1 | | |
| SL003648 | GDI2 | Rab GDPdissociation | 2.72E−10 | −1.07 |
| | | inhibitor beta | | |
| SL003302 | CCL23 | C-C motif chemokine 23 | 4.51E−10 | 1.65 |
| SL005694 | PRDX6 | Peroxiredoxin-6 | 4.72E−10 | −1.00 |
| SL003655 | TKT | Transketolase | 4.72E−10 | −1.09 |
| SL006910 | CTSV | Cathepsin L2 | 4.73E−10 | −1.51 |
| SL012774 | CRELD1 | Cysteine-rich with EGF- | 6.89E−10 | 1.28 |
| | | like domain protein 1 | | |
| SL004812 | TPI1 | Triosephosphate isomerase | 1.66E−09 | −1.26 |
| SL004921 | NME2 | Nucleoside diphosphate | 1.66E−09 | −1.14 |
| | | kinase B | | |
| SL002525 | C2 | Complement C2 | 1.74E−09 | 0.65 |
| SL003542 | EHMT2 | Histone-lysine N- | 4.18E−09 | −0.69 |
| | | methyltransferase EHMT2 | | |
| SL009324 | FSTL3 | Follistatin-related protein 3 | 5.56E−09 | 1.51 |
| SL003301 | CCL23 | Ck-beta-8-1 | 5.70E−09 | 1.29 |
| SL002621 | MDK | Midkine | 1.59E−08 | −2.65 |
| SL014069 | PCSK7 | Proprotein convertase | 2.81E−08 | 0.90 |
| | | subtilisin/kexin type 7 | | |
| SL017289 | UBB | PolyUbiquitin K48-linked | 3.17E−08 | −0.97 |
| SL010524 | WNK3 | Serine/threonine- protein | 3.51E−08 | −0.86 |
| | | kinase WNK3 | | |
| SL000455 | JUN | Transcription factor AP-1 | 4.50E−08 | 0.64 |
| SL004145 | TNFRSF 14 | Tumor necrosis factor | 5.02E−08 | 0.46 |
| | | receptor superfamily | | |
| | | member 14 | | |
| SL003522 | ERP29 | Endoplasmic reticulum | 5.14E−08 | −0.85 |
| | | resident protein 29 | | |
| SL008178 | DPT | Dermatopontin | 7.81E−08 | −0.89 |
| SL010288 | CA6 | Carbonic anhydrase 6 | 3.01E−07 | −2.42 |
| SL003755 | MCL1 | Induced myeloid leukemia | 8.54E−07 | 0.70 |
| | | cell differentiation protein | | |
| | | Mcl-1 | | |
| SL003300 | CCL16 | C-C motifchemokine 16 | 9.47E−07 | −1.76 |
| SL000022 | FGA FGB | D-dimer | 9.62E−07 | 1.50 |
| | FGG | | | |
| SL004821 | S100A4 | Protein S100-A4 | 9.62E−07 | −0.93 |
| SL000321 | C5 C6 | Complement C5b-C6 | 1.11E−06 | 0.37 |
| | | complex | | |
| SL006777 | FETUB | Fetuin-B | 1.76E−06 | −1.13 |
| SL007306 | FAM3B | Protein FAM3B | 1.90E−06 | −0.94 |
| SL004097 | SMAD3 | Mothers against | 2.17E−06 | 0.71 |
| | | decapentaplegic homolog 3 | | |
| SL013969 | KYNU | Kynureninase | 2.54E−06 | 1.03 |
| SL000382 | CKB CKM | Creatine kinase M- | 3.55E−06 | −2.65 |
| | | type: Creatine kinase B-type | | |
| | | heterodimer | | |
| SL004919 | PRDX1 | Peroxiredoxin-1 | 3.71E−06 | −0.86 |
| SL004338 | FGF20 | Fibroblast growth factor 20 | 6.65E−06 | −0.33 |
| SL010388 | PRSS2 | Trypsin-2 | 1.46E−05 | 1.61 |
| SL008486 | LGALS9 | Galectin-9 | 1.88E−05 | 0.63 |
| SL000310 | C1R | Complement Clr | 1.99E−05 | 1.05 |
| | | subcomponent | | |
| SL014270 | CD300C | CMRF35-like molecule 6 | 2.16E−05 | 0.75 |
| SL000645 | MMP10 | Stromelysin-2 | 3.64E−05 | 1.23 |
| SL007108 | IRF1 | Interferon regulatory | 7.20E−05 | 0.21 |
| | | factor 1 | | |
| SL010619 | TPSG1 | Tryptase gamma | 7.76E−05 | −0.47 |
| SL003189 | CCL19 | C-C motifchemokine 19 | 0.000102049 | 1.37 |
| SL002722 | CD38 | ADP-ribosyl cyclase/cyclic | 0.000190617 | 0.21 |
| | | ADP-ribose hydrolase 1 | | |
| SL000383 | CKM | Creatine kinase M-type | 0.000433221 | −1.52 |
| SL004359 | NTF3 | Neurotrophin-3 | 0.000636868 | −0.49 |
| SL000325 | C9 | Complement component C9 | 0.001072455 | 0.60 |
| SL006705 | PFDN5 | Prefoldin subunit 5 | 0.001304314 | −0.60 |
| SL012822 | PRSS22 | Brain-specific serine | 0.005810436 | 0.61 |
| | | protease 4 | | |

The biomarkers in Table 1 are human proteins. However, this disclosure can comprise homologs thereof from other animals. Thus, while in many embodiments the subject is a human, the disclosure also encompasses embodiments where the subject is a non-human primate, dog, cat, rodent, or other mammal of veterinary or medical model interest. In one embodiment, the subject is a young human, such as a pediatric patient, e.g., less than 20 years old.

Table 1 provides markers that have been determined to be newly associated with sepsis. As discussed in more detail below, Table 5 discloses 111 protein biomarkers determined to associate (e.g., are differentially expressed) in subjects with sepsis. Table 6 lists biomarkers that have been previously associated with sepsis. The markers that are disclosed in Table 5 and which are not listed in Table 6 are newly associated with sepsis, now recited in Table 1.

While the status of the disclosed proteins in Table 1 as markers for sepsis were not heretofore know, the proteins themselves are known and identifiable by the indicated proteins names and Entrez gene names. A person of ordinary skill in the art can readily identify the indicated proteins and homologs thereof based on the current databases of protein names, structures, and canonical functions. Based on the present disclosure that also illuminates the status of these proteins as markers of sepsis, a person of ordinary skill in the art can generate and use affinity reagents, as described herein, that can detect such proteins to perform the methods disclosed herein.

While the method is generally described in terms of a single biomarker, the disclosure encompasses the use of a plurality (e.g., a panel) of different biomarkers wherein the method is performed, e.g., in batch, for each biomarker in parallel. Thus, in some embodiments, the method comprises contacting the sample obtained from the subject with a plurality of different affinity reagents that bind to two or more biomarkers for sepsis. For example, the method encompasses using a plurality of different affinity reagents that bind to a panel of about 2 to about 150, about 2 to about 125, about 2 to about 100, about 2 to about 90, about 2 to about 80, about 2 to about 70, about 2 to about 60, about 2 to about 50, about 2 to about 40, about 2 to about 30, about 2 to about 20, about 2 to about 10, about 10 to about 150, about 10 to about 125, about 10 to about 100, about 10 to about 90, about 10 to about 80, about 10 to about 70, about 10 to about 60, about 10 to about 50, about 10 to about 40, about 10 to about 30, about 10 to about 20, about 20 to about 150, about 20 to about 125, about 20 to about 100, about 20 to about 90, about 20 to about 80, about 20 to about 70, about 20 to about 60, about 20 to about 50, about 20 to about 40, about 20 to about 30, about 30 to about 150, about 30 to about 125, about 30 to about 100, about 30 to about 90, about 30 to about 80, about 30 to about 70, about 30 to about 60, about 30 to about 50, about 30 to about 40, about 40 to about 150, about 40 to about 125, about 40 to about 100, about 40 to about 90, about 40 to about 80, about 40 to about 70, about 40 to about 60, about 40 to about 50, about 50 to about 150, about 50 to about 125, about 50 to about 100, about 50 to about 75, about 75 to about 150, about 75 to about 125, about 75 to about 100, about 100 to about 150, about 100 to about 125, about 125 to about 150 biomarkers, or any range therein. For example, the method encompasses using a plurality of different affinity reagents that bind to a panel of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more biomarkers.

The additional biomarker(s) for sepsis can be previously known (e.g., as selected from the biomarkers listed in Table 6) or heretofore unknown (e.g., as selected from the biomarkers listed in Table 1). In some embodiments, the method comprises sample obtained from the subject with a panel of different affinity reagents that bind any number of a plurality of the 111 biomarkers listed in Table 5. In some embodiments, the panel includes one or more of the biomarkers listed in Table 2, which discloses the top 20 protein biomarkers associated with sepsis as determined in the study described in Example 1. In some embodiments, the biomarker (or a plurality of biomarkers in the panel) is selected from the biomarkers disclosed in Table 3. Increased expression of these biomarkers indicates the inflammatory condition in the subject is sepsis.

In some embodiments, the biomarker (or at least one of the plurality of biomarkers) is a protease, such as BMP1, CTSF, CTSV, MMP10, PRSS2, and/or TPSG1. In some embodiments, the biomarker (or at least one of the plurality of biomarkers) is an intracellular protein, such as ANK2, CRELD1, EHMT2, ERP29, MCL1, MED1, and/or PIAS4. In some embodiments, the biomarker (or at least one of the plurality of biomarkers) is a growth factor, such as FGF18, FGF20, NTF3, and/or PTN. In yet further embodiments, the biomarker (or at least one of the plurality of biomarkers) is WIF1. In some embodiments, in addition to the novel biomarker described above, the method further comprises contacting the sample with one or more affinity reagents that bind to at least one of THPO, PLAUR, IL-22, and/or EPO, or any combination thereof.

The method can further comprise obtaining the biological sample from the subject. The biological sample can be any biological sample that contains circulating protein biomarkers, such as blood and derivatives of blood (e.g. plasma and serum).

The term “affinity reagent” refers to any molecule or receptor capable of specifically binding a target molecule or antigen, such as a specific sepsis biomarker. In many embodiments the target molecule or antigen is a protein biomarker.

As used herein, the term “specifically bind” or variations thereof refer to the ability of affinity reagent to bind to the antigen of interest (e.g., sepsis biomarker), without significant binding to other molecules, under standard conditions known in the art. For example, the affinity reagent can bind to other peptides, polypeptides, or proteins, but with lower affinity. However, the affinity reagent preferably does not substantially cross-react with other antigens (e.g., biomarkers). For example, in some embodiments, the affinity reagent specifically binds with an affinity or Kₐ (i.e., an equilibrium association constant of a particular binding interaction with units of 1/M) equal to or greater than 10⁵ M⁻¹, while not significantly associating or uniting with any other molecules or components in a sample. The affinity reagent can be classified as a “high affinity” affinity reagent or a “low affinity” affinity reagent. “High affinity” affinity reagents refer to affinity reagents with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” affinity reagents refer to affinity reagents with a Kₐ of up to 10⁷ M⁻¹, up to 10⁶ M⁻¹, up to 10⁵ M. Alternatively, affinity can be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domain with stronger binding to a target antigen than a wild type (or parent) binding domain. For example, enhanced affinity may be due to a Kₐ (equilibrium association constant) for the target antigen that is higher than the wild type binding domain, or due to a K_{d} (dissociation constant) for the target antigen that is less 10 than that of the wild type binding domain, or due to an off-rate (K_{off}) for the target antigen that is less than that of the wild type binding domain. A variety of assays are known for identifying binding domains of the present disclosure that specifically bind a particular target, as well as determining binding domain affinities, such as Western blot, ELISA, and Biacore® analysis (see also, e.g., Scatchard et al., Ann. N.Y. Acad. Sci. 51:660, 1949; and U.S. Pat. Nos. 5,283,173, 5,468,614, or the equivalent).

In some embodiments, the affinity reagent can be an antibody, or an antibody fragment or derivative. The term “antibody” is used herein in the broadest sense and encompasses various antibody structures derived from any antibody-producing mammal (e.g., mouse, rat, rabbit, and primate including human), and which specifically bind to an antigen of interest. An antibody fragment specifically refers to an intact portion or subdomain of a source antibody that still retains antigen-biding capability. An antibody derivative refers to a molecule that incorporates one or more antibodies or antibody fragments. Typically there is at least some additional modification in the structure of the antibody or fragment thereof, or in the presentation or configuration of the antibody or fragment thereof. Exemplary antibodies of the disclosure include polyclonal, monoclonal and recombinant antibodies. Exemplary antibodies or antibody derivatives of the disclosure also include multispecific antibodies (e.g., bispecific antibodies); humanized antibodies; murine antibodies; chimeric, mouse-human, mouse-primate, primate-human monoclonal antibodies; and anti-idiotype antibodies.

As indicated, an antibody fragment is a portion or subdomain derived from or related to a full-length antibody, preferably including the complementarity-determining regions (CDRs), antigen binding regions, or variable regions thereof, and antibody derivatives refer to further structural modification or combinations in the resulting molecule. Illustrative examples of antibody fragments or derivatives encompassed by the present disclosure include Fab, Fab′, F(ab)₂, F(ab′)₂ and Fv fragments, diabodies, single-chain antibody molecules, V_{H}H fragments, V_{NAR} fragments, multispecific antibodies formed from antibody fragments, nanobodies and the like. For example, an exemplary single chain antibody derivative encompassed by the disclosure is a “single-chain Fv” or “scFv” antibody fragment, which comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Another exemplary single-chain antibody encompassed by the disclosure is a single-chain Fab fragment (scFab).

Antibody fragments and derivatives that recognize specific epitopes can be generated by any technique known to those of skill in the art. For example, Fab and F(ab′)₂ fragments of the disclosure can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies, or fragments or derivatives thereof, of the present disclosure can also be generated using various phage display methods known in the art. Finally, the antibodies, or fragments or derivatives thereof, can be produced recombinantly according to known techniques.

It will be apparent to the skilled practitioner that the affinity reagents can comprise binding domains other than antibody-based domains, such as peptidobodies, antigen-binding scaffolds (e.g., DARPins, HEAT repeat proteins, ARM repeat proteins, tetratricopeptide repeat proteins, and other scaffolds based on naturally occurring repeat proteins, etc. [see, e.g., Boersma and Pluckthun, Curr. Opin. Biotechnol. 22:849-857, 2011, and references cited therein, incorporated herein by reference]), and aptamers, which include a functional biomarker-binding domain.

In some embodiments, the affinity reagent is an aptamer. An aptamer is a type of oligonucleotide or peptide/protein-based affinity reagent. In some embodiments, the aptamer is an oligonucleotide (e.g., DNA, RNA, or XNA) that usually are short strands that are at least sufficient in length to adopt a conformation conferring specific antigen (e.g., biomarker) binding abilities. In other embodiments, the aptamers comprise peptide structures. In yet other embodiments, the aptamer is a longer peptide, i.e., protein, such as an affimer, which is a small and highly stable protein engineered to display peptide loops that confer highly specific binding properties. Specific binding is conferred by electrostatic interactions, hydrophobic interactions, and complementary shapes between the aptamer and the target biomarker antigen.

Illustrative, non-limiting configurations of the affinity reagents will now be discussed. While this discussion is presented in the context of aptamer, it will be appreciated that the present disclosure encompasses all affinity reagents (e.g., antibodies and fragments or derivatives thereof).

The affinity reagent (e.g., antibody, antibody fragment or derivative, aptamer, and the like) can be immobilized to a surface or solid support. The surface or solid support can be or comprise a particle (including, but not limited to an agarose or latex bead or particle or a magnetic particle), a bead, a nanoparticle, a polymer, a substrate, a slide, a coverslip, a plate, a dish, a well, a membrane, and/or a grating. The solid support can include many different materials including, but not limited to, polymers, plastics, resins, polysaccharides, silicon or silica based materials, carbon, metals, inorganic glasses, and membranes. In some embodiments, the affinity reagent (e.g., antibody, antibody fragment or derivative, aptamer, and the like) is immobilized to a bead. In further embodiments, the affinity reagent (e.g., antibody, antibody fragment or derivative, aptamer, and the like) is immobilized to the bead via a tether. The tether can be cleavable, e.g., by enzymatic action. In other embodiments, the tether is susceptible to light cleavage. Exemplary configurations for affinity reagents, and especially aptamer affinity reagents, encompassed by this disclosure are disclosed in more detail in Ruscito, A., DeRosa, M. C., Small-Molecule Binding Aptamers: Selection Strategies, Characterization, and Applications, Front Chem. 4:14, 2016; doi: 10.3389/fchem.2016.00014, incorporated herein by reference in its entirety.

The affinity reagent (e.g., antibody, antibody fragment or derivative, aptamer, and the like) can also comprise a detectable label and/or comprise the ability to generate a detectable signal (e.g. by catalyzing a reaction converting a compound to a detectable product). Detectable labels can comprise, for example, a light-absorbing dye, a fluorescent dye, or a radioactive label. Detectable labels, methods of detecting them, and methods of incorporating them into an affinity reagent are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence; or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies), The detectable label can be linked by covalent or non-covalent means to the affinity reagent (e.g., antibody, antibody fragment or derivative, aptamer, and the like). Alternatively, a detectable label can be linked such as by directly labeling a molecule that achieves binding to the affinity reagent (e.g., antibody, antibody fragment or derivative, aptamer, and the like) via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the affinity reagent (e.g., antibody, antibody fragment or derivative, aptamer, and the like) is labeled with a fluorescent compound. When the fluorescently labeled affinity reagent is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, pefidenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluroescein (JOE or N,N,N′,N′-tetramethyl-6carboxyrhodamine (TAMRA, or T), 6-carboxy-X-rhodamine (ROX or R), 5-carboxyrhodamine-6G (R6G5 or G5), 6-carboxyrhodamine-60 (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes.

In some embodiments, a detectable label can be a radiolabel including, but not limited to 3H, 125I, 35S, 14C, 32P, and 33P.

In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In some embodiments, a detectable label is a chemiluminescent label, including, but not limited to lucigenin, luminol, luciferin, isoluminol, theromatic acridinium ester, imidazole, acridinium salt and oxalate ester.

In some embodiments, a detectable label can be a spectral colorimetric label including, but not limited to colloidal gold or colored glass or plastic (e.g., polystyrene, polypropylene, and latex) beads.

In some embodiments, affinity reagent (e.g., antibody, antibody fragment or derivative, aptamer, and the like) can also be labeled with a detectable tag, such as c-Myc, HA, VSV-G, HSV, FLAG, V5, HIS, or biotin. Other detection systems can also be used, for example, a biotin-streptavidin system. In this system, the affinity reagent (e.g., antibody, antibody fragment or derivative, aptamer, and the like) that is reactive with (i.e. specific for) the biomarker of interest is biotinylated. Quantity of biotinylated affinity reagent bound to the biomarker is determined using a streptavidin-peroxidase conjugate and a chromagenic substrate. Such streptavidin peroxidase detection kits are commercially available.

An affinity reagent (e.g., antibody, antibody fragment or derivative, aptamer, and the like) can also be detectably labeled using fluorescence emitting metals such as 152Eu, or others of the lanthanide series. These metals can be attached to the antibody reagent using such metal chelating groups as diethylenetriaminepentaacetic acid (DTPA) or ethylenediaminetetraacetic acid (EDTA).

In some embodiments, the step of detecting the differential expression of the biomarker by detecting binding of the affinity reagent to the biomarker comprises comparing the binding level to a reference standard. The reference standard can be derived from a subject without sepsis, such as a health subject or, in some embodiments, a subject with infection-negative systemic inflammation (INSI) or other conditions. The reference standard can be an established quantitative value of binding of the affinity reagent to protein in a sample obtained from one or more reference individuals (i.e., individuals without sepsis). In some embodiments, the method further comprises determining the reference standard value of binding. The differential expression of the biomarker by detecting binding of the affinity reagent to the biomarker can be determined when the affinity reagent binds to the biomarker at a level that is different than a level of binding from a sample derived from the reference individual(s). In some embodiments, the difference in binding levels is significant according to standard statistical approaches. When a difference in binding level compared to the reference level is determined, differential expression of the biomarker is inferred, which in turn indicates sepsis in the subject. The difference can be reflective of a relative increase or decrease of the expression compared to the reference standard. For example, the relative increase or decrease of expression for each biomarker as it relates to sepsis (as compared to INSI) is indicated in, e.g., Table 5.

As indicated above, the method can be performed utilizing a panel of different biomarkers for sepsis, thus incorporating a plurality of different affinity reagents that specifically bind the different members of the biomarker panel. For example, Example 1 describes the use of a panel of aptamer affinity reagents on a SOMAscan® platform available from SOMAlogic (Boulder, Colo.). The disclosure encompasses embodiments of panels that incorporate such exemplary aptamers and related system components.

As indicated, upon detection of differential expression of sepsis biomarkers in a subject, the subject is determined to have sepsis. An advantage of the disclosed method is identification of sepsis biomarkers and sepsis diagnosis of a subject can occur earlier than is currently possible or typically conducted, resulting in sepsis treatment at an earlier time point during progression of the sepsis condition. Earlier diagnosis and administration of treatment yields increased likelihood for treatment success and subject recovery from the sepsis condition. An additional advantage of the ability to effectively diagnose for sepsis includes decreased false-positives and resulting misdiagnosis, which leads to unnecessary costs and health effects associated with testing, treatment, and hospitalized care for a sepsis condition the subject does not exhibit.

Thus, in some embodiments, the method further comprises treating the subject indicated as having sepsis for the sepsis condition.

As used herein, the term “treat” refers to medical management of a disease, disorder, or condition (e.g., sepsis) of a subject (e.g., a human or non-human mammal, such as another primate, horse, dog, mouse, rat, guinea pig, rabbit, and the like). Treatment can encompass any indicia of success in the treatment or amelioration of the disease or condition (e.g., sepsis), including any parameter such as abatement, remission, diminishing of symptoms or making the disease or condition more tolerable to the subject, slowing in the rate of degeneration or decline, or making the degeneration or sepsis less debilitating. The treatment or amelioration of symptoms can be based on objective or subjective parameters, including the results of an examination by a physician. Accordingly, the term “treating” includes the administration of appropriate therapeutic compositions to alleviate, or to arrest or inhibit development of the symptoms or conditions associated with the disease or condition (e.g., sepsis). The term “therapeutic effect” refers to the amelioration, reduction, or elimination of the disease or condition, symptoms of the disease or condition, or side effects of the disease or condition in the subject. The term “therapeutically effective” refers to an amount of the composition that results in a therapeutic effect and can be readily determined. Specifically in the context of sepsis, the term treat can encompass administration of therapeutic interventions (e.g., agents) for reducing inflammation, reducing pain associated with inflammation, controlling body temperature, maintaining blood pressure, or reducing the likelihood of recurrence, compared to not having the treatment.

In some embodiments, the method comprises administering the treatment multiple times as the sepsis is monitored.

In another aspect, the disclosure provides a method of characterizing an inflammatory state in a subject that differentiates between sepsis and infection-negative systemic inflammation (INSI).

In some embodiments, the method comprises contacting a biological sample obtained from the subject with at least one affinity reagent that specifically binds to at least one biomarker selected from Table 5, wherein differential expression of the biomarker indicates whether the inflammatory condition is sepsis or INSI. A net increase in the expression any one or more of the biomarkers listed in Table 5 with a positive value in the “Log 2 fold change (sepsis vs INSI)” column relative to a reference level indicates that the subject has sepsis and not INSI. In contrast, a net increase in the expression any one or more of the biomarkers listed in Table 5 with a negative value in the “Log 2 fold change (sepsis vs INSI)” column relative to a reference level indicates that the subject has INSI and not sepsis. In some embodiments, the at least one affinity specifically binds to at least one biomarker selected from the biomarkers set forth in Table 1, Table 2, or Table 3.

In some embodiments, the method comprises contacting a biological sample obtained from the subject with an affinity reagent that specifically binds to a biomarker selected from the biomarkers disclosed in Table 3, and detecting differential expression of the biomarker by detecting binding of the affinity reagent to the biomarker. Increased expression of the biomarker indicates the inflammatory condition in the subject is sepsis and not INSI.

The method further comprises treating the subject as appropriate for sepsis or INSI, based on the characterization of the inflammatory condition.

In another aspect, the disclosure provides a method of monitoring sepsis in a subject. The monitoring comprises performing an embodiment of the method as described above at multiple time points. With monitoring over time, the practitioner can determine whether sepsis is persisting or has been overcome. In some embodiments, the detection of differential expression of the biomarker is to some degree quantitative and reflects relative severity of the sepsis. Thus, monitoring over time can permit determination of the progression of the sepsis condition over time (e.g., whether the sepsis condition is growing more or less severe.) In some embodiments, a reduction in detected differential expression of the biomarker indicates an amelioration of sepsis in the subject, whereas an increase in the detected differential expression of the biomarker indicates an increased severity of sepsis in the subject. In this regard, “increased” and “decreased” differential expression refers to the magnitude of the difference of expression from a reference standard expression level. For example, a reduction in detected differential expression of the biomarker compared to the expression level in a healthy subject (or other subject without sepsis) indicates an amelioration of sepsis in the subject.

In some embodiments, at least one of the two or more time points is during or after administration of a treatment for sepsis to the subject. Accordingly, the monitoring approach is applicable to assessing the efficacy of a treatment of sepsis. A reduction of detected differential expression of the biomarker over time during or after treatment indicates that the treatment is effective to control or reduce sepsis. Illustrative treatments strategies are discussed in more detail above.

In another aspect, the disclosure provides a kit for detection and/or monitoring of sepsis from a biological sample.

The kit can comprise any of the affinity reagents described herein. In additional embodiments, the kit also comprises indicia (e.g., written text and/or diagrams) that guide how to perform an embodiment of the methods described above.

### General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are provided for the purpose of illustrating, not limiting, the disclosure.

### Example 1

This Example describes a retrospective study cohort study of subjects conducted to identify reliable markers for sepsis.

### Results

Screen

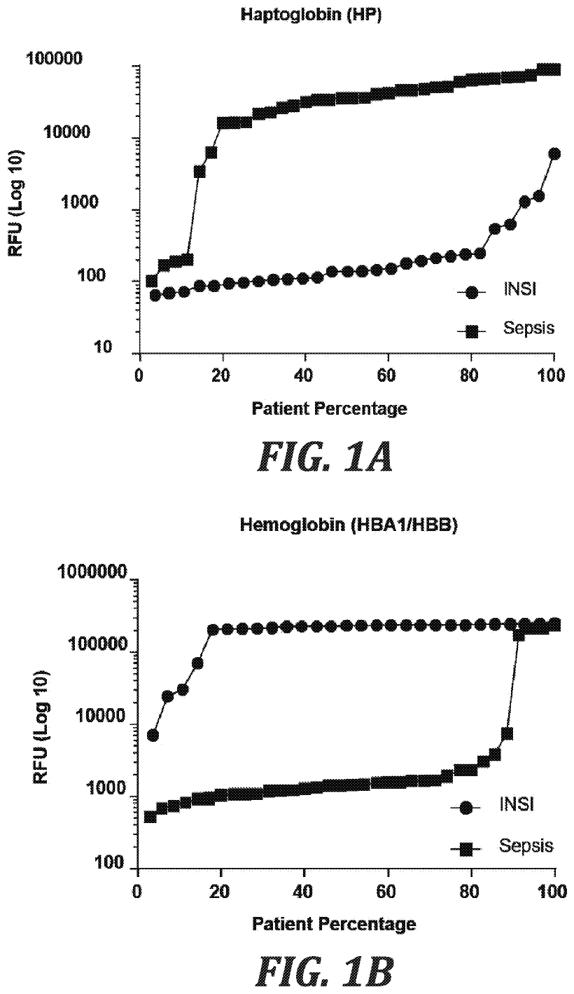

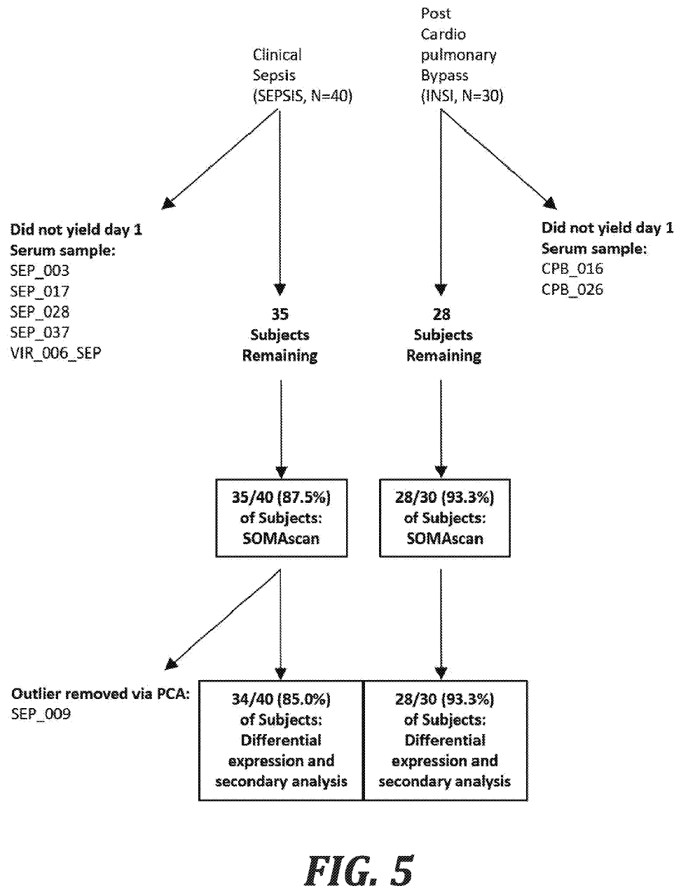

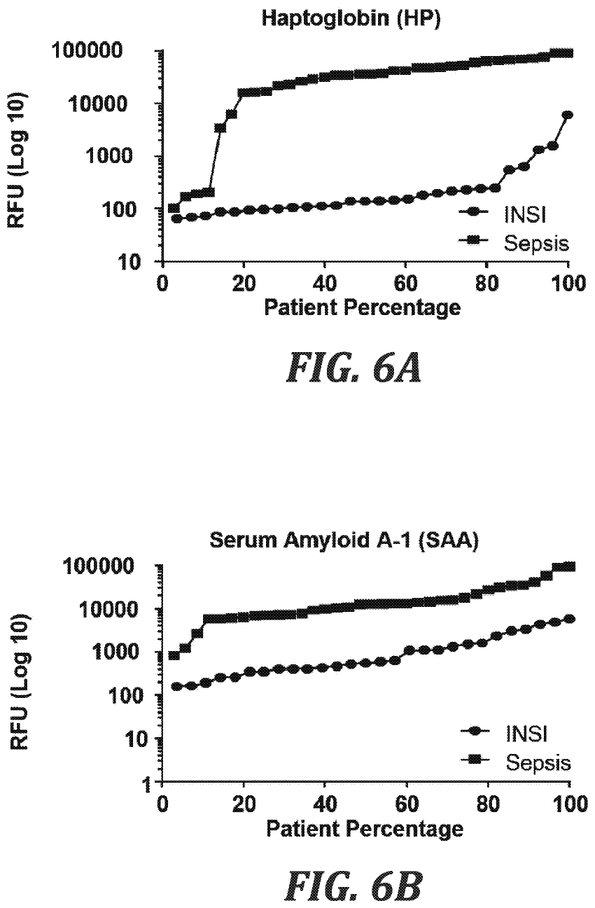

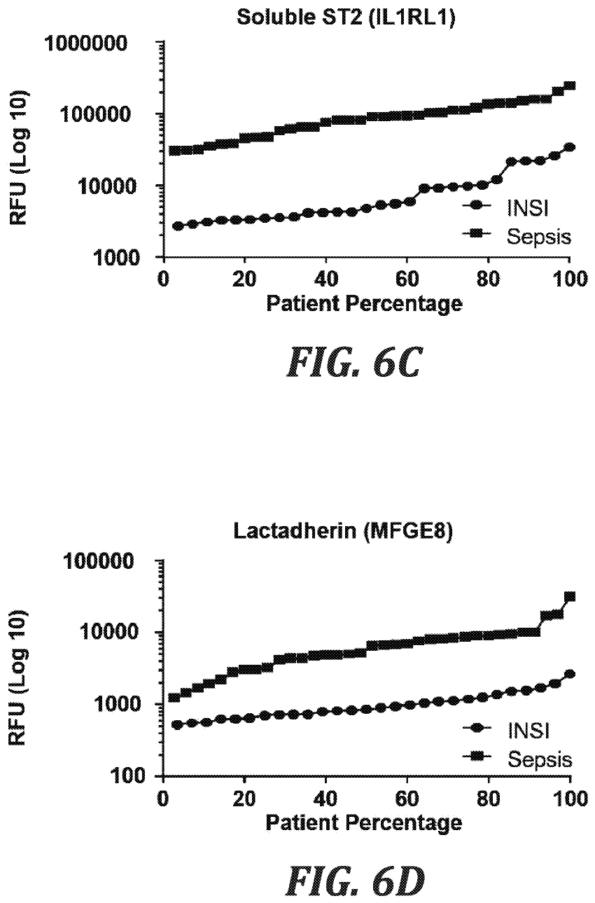

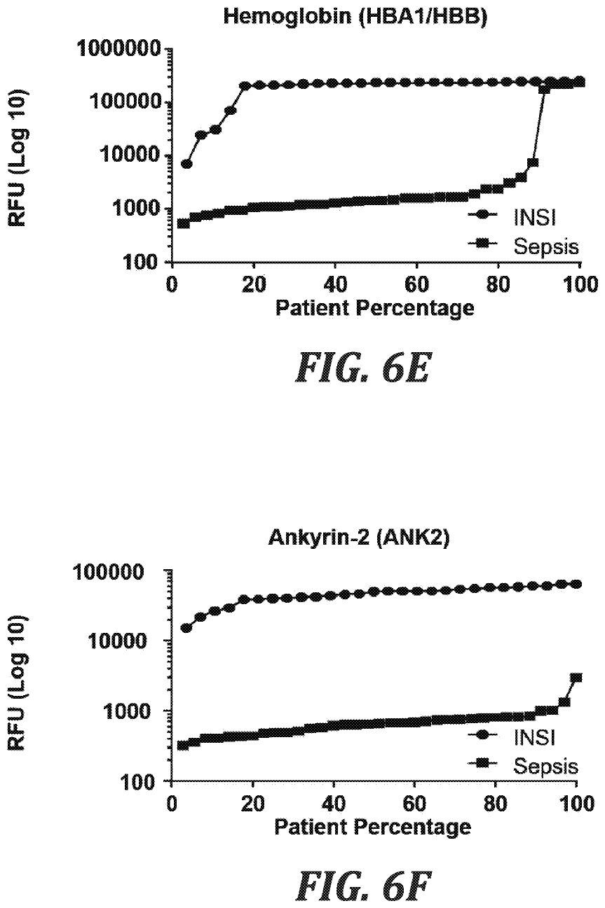

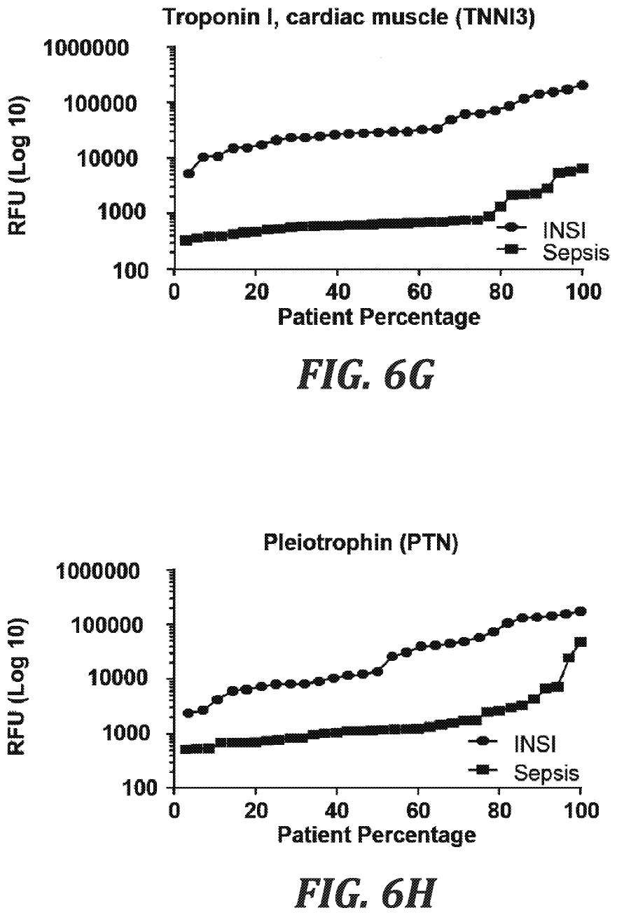

Specific differences regarding the clinical characteristics of the two patient groups are found in Table 4 (see “Additional Tables” section for Tables 4-9), and a consort diagram depicting inclusion criteria for subsequent analysis following the SOMAscan proteomic analysis is found in FIG. 5. One hundred eleven proteins (Table 2 [top 20 proteins] and Table 5 [111 proteins, represented by 112 SOMAmers]) differentially expressed between the sepsis and INSI patients were identified. To provide an alternative visualization of the differential protein expression between the sepsis and INSI groups, empirical cumulative density function plots were constructed for four of the top upregulated (FIGS. 1A and 1B, and FIGS. 6A-6D) and downregulated (FIGS. 1A and 1B, and FIGS. 6E-6H) proteins between the two groups.

Importantly, 55 of the 111 proteins had well-documented prior associations with sepsis, and 19 of these proteins were previously evaluated in pediatric sepsis patients (Table 6), validating the aptamer proteomics methodology.

TABLE 2
the top twenty differentially expressed proteins. Sepsis:
clinically overt sepsis; INSI: infection negative
systemic inflammation caused by cardiopulmonary bypass;
LIMMA: linear models for microarray data.

| | | | | Log2 Fold |
|---|---|---|---|---|
| | | Entrez | | Change |
| | | Gene | Adjusted | (SEPSIS |
| SOMAmer | Protein Name | Symbol | P-Value* | vs. INSI) |
| SL014896 | Ankyrin-2 | ANK2 | 6.84E−47 | −5.89 |
| SL001761 | Troponin I, cardiac | TNNI3 | 2.24E−25 | −5.21 |
| | muscle | | | |
| SL004146 | Interleukin-1 receptor- | IL1RL1 | 1.83E−19 | 3.54 |
| | like 1 | | | |
| SL003309 | Lipopoly saccharide- | LBP | 1.98E−19 | 2.13 |
| | binding protein | | | |
| SL000437 | Haptoglobin | HP | 2.25E−16 | 6.71 |
| SL000836 | Hemoglobin | HBA1 | 1.15E−15 | −6.18 |
| | | HBB | | |
| SL003770 | Secreted frizzled- | SFRP1 | 1.16E−15 | −3.94 |
| | related protein 1 | | | |
| SL000055 | Cadherin-1 | CDH1 | 1.47E−15 | −1.34 |
| SL007631 | Sclerostin | SOST | 2.05E−15 | −1.09 |
| SL006523 | Lactadherin | MFGE8 | 2.35E−14 | 2.83 |
| SL008381 | Cathepsin F | CTSF | 5.91E−14 | −0.93 |
| SL000572 | Serum amyloid A-1 | SAA1 | 3.49E−13 | 3.94 |
| | protein | | | |
| SL008023 | Hyaluronan and | HAPLN1 | 3.94E−13 | −1.61 |
| | proteoglycan link | | | |
| | protein 1 | | | |
| SL004652 | Wnt inhibitory factor 1 | WIF1 | 1.09E−12 | −0.85 |
| SL010328 | Mediator of RNA | MED1 | 1.28E−12 | −1.21 |
| | polymerase II | | | |
| | transcription subunit 1 | | | |
| SL004152 | Interleukin-18 | IL18R1 | 1.51E−12 | 1.05 |
| | receptor 1 | | | |
| SL004336 | Fibroblast growth | FGF18 | 1.51E−12 | −1.63 |
| | factor 18 | | | |
| SL000462 | Insulin-like growth | IGFBP1 | 1.51E−12 | −2.42 |
| | factor-binding | | | |
| | protein 1 | | | |
| SL002704 | Pleiotrophin | PTN | 1.77E−12 | −3.95 |
| SL002508 | Interleukin-18-binding | IL18BP | 1.86E−12 | 2.46 |
| | protein | | | |

*Benjamini-Hochberg multiple testing correction from LIMMA

Clinical and Demographic Covariate Analysis

Expression levels of the differentially expressed 111 proteins were not significantly confounded by demographic or clinical variables. Sex, age, immune competency status, cancer diagnosis, or viral infections in the sepsis patients were assessed for the expression of the 1,305 proteins evaluated by the Boruta algorithm. Stromal cell-derived factor 1 (CXCL12), ferritin light chain (FTH1/FTL), hyaluronan and proteoglycan link protein 1 (HAPLN1), serum albumin (ALB), and galectin-9 (LGALS9) were the only proteins confounded by any of these variables (Table 7). Therefore, they were not considered relevant in further analyses.

Identification of Proteins with Probable Associations with Sepsis

Assessment of the differentially expressed proteins most strongly associated with sepsis required weighted gene correlation network analysis (WGCNA). WGCNA is an unbiased data-driven approach that searches for correlations between changes in groups of proteins (protein co-expression modules) and clinical traits. Clinical traits recorded in the case report forms obtained from all patients in the study were utilized.

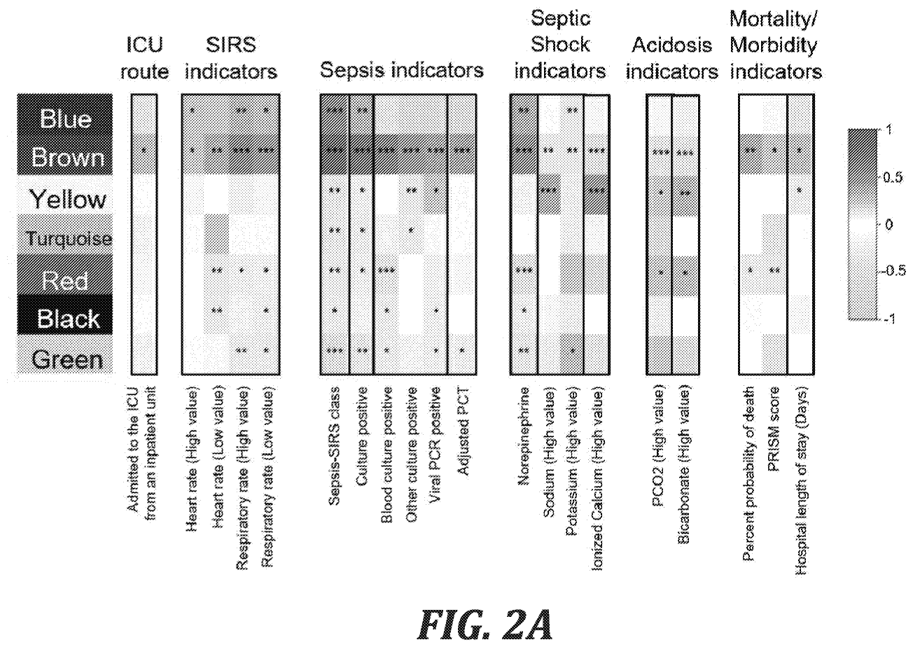

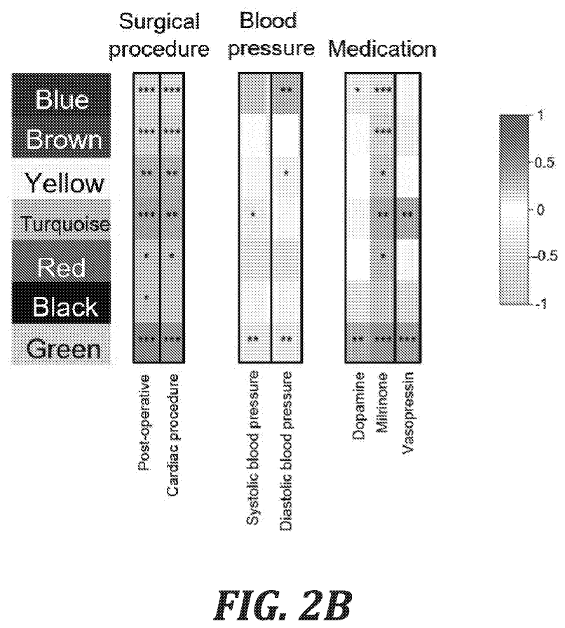

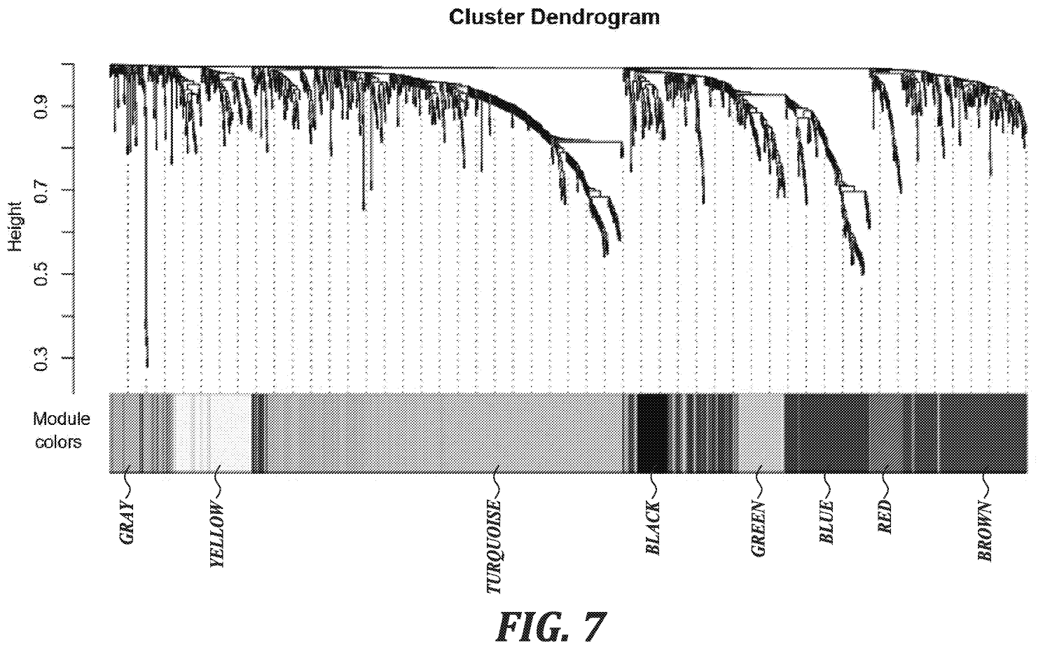

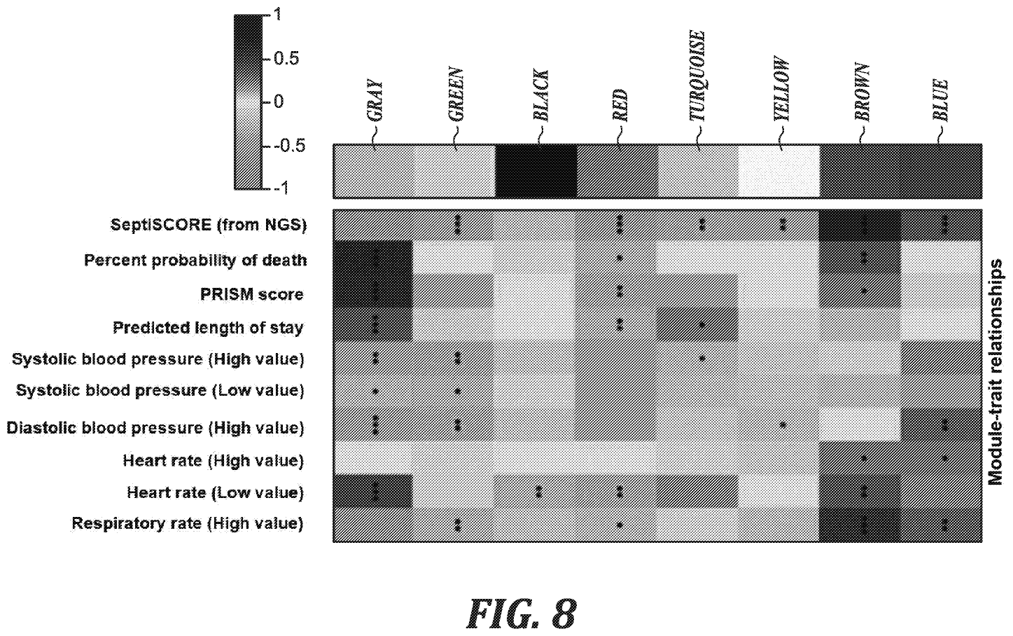

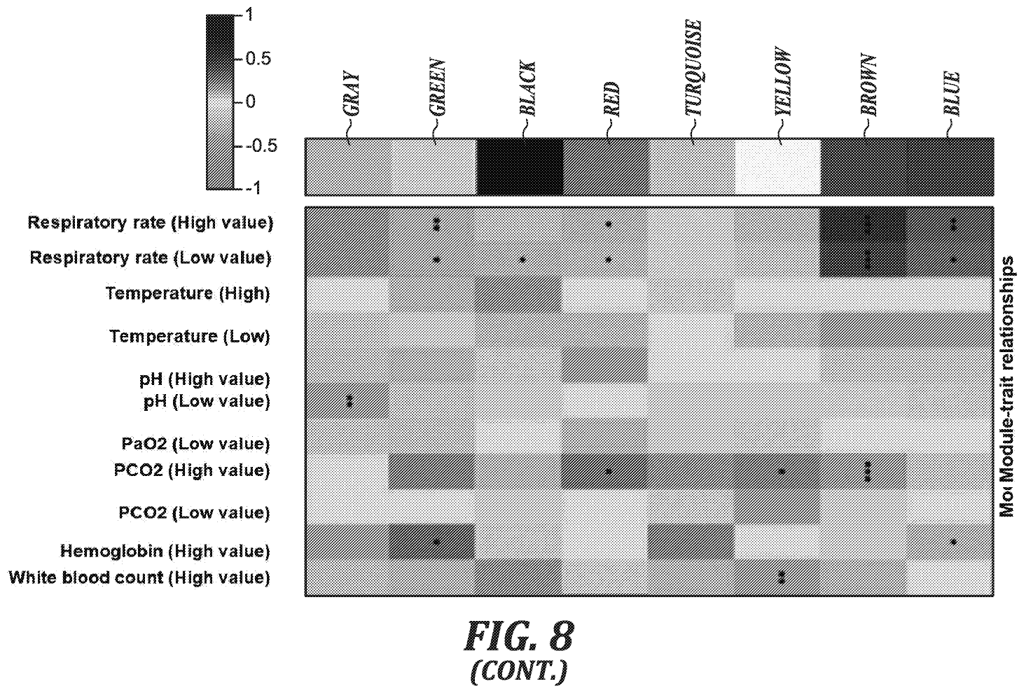

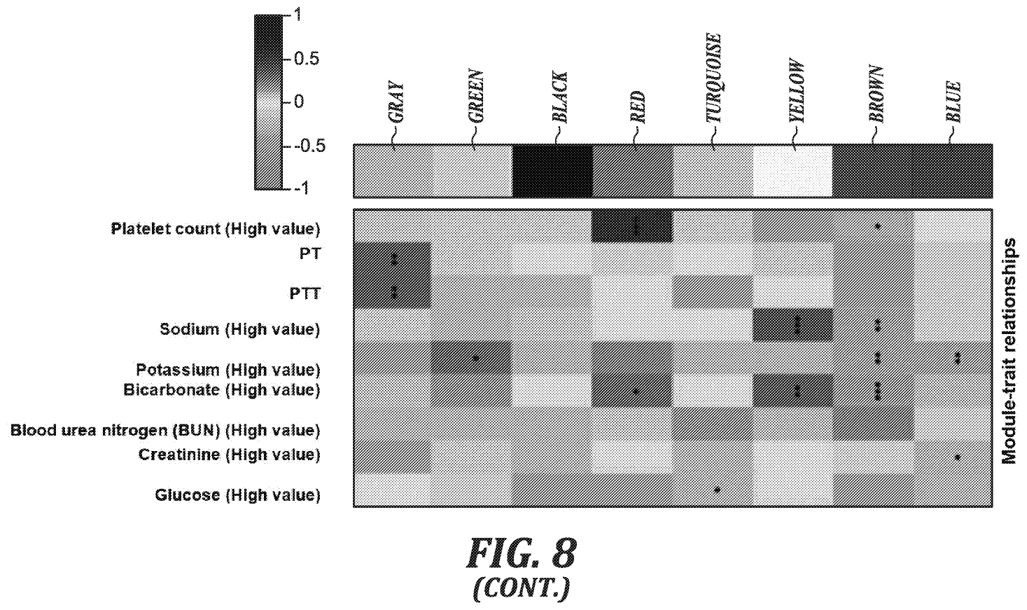

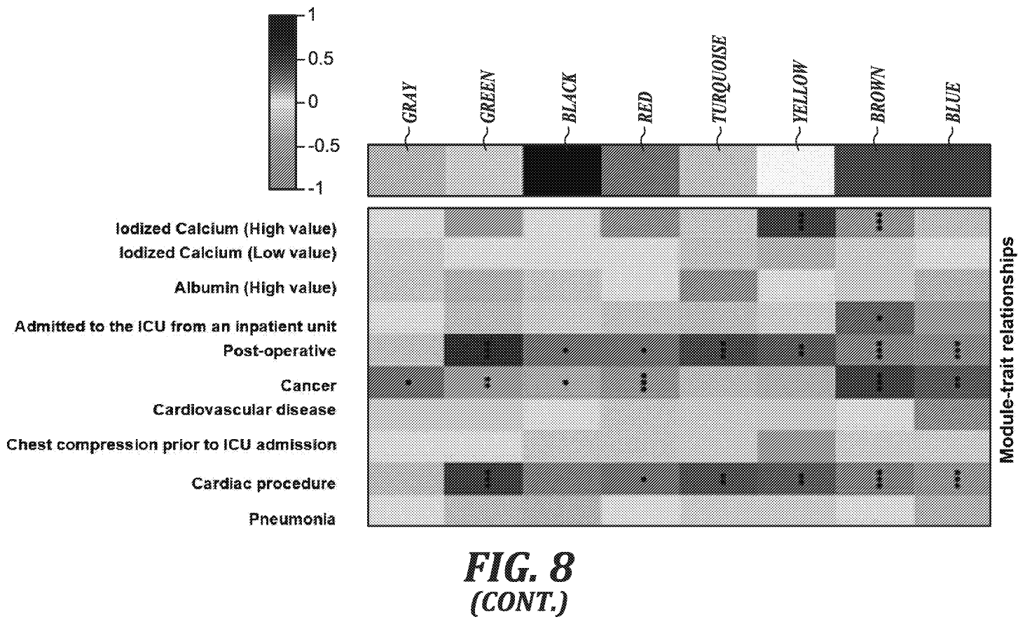

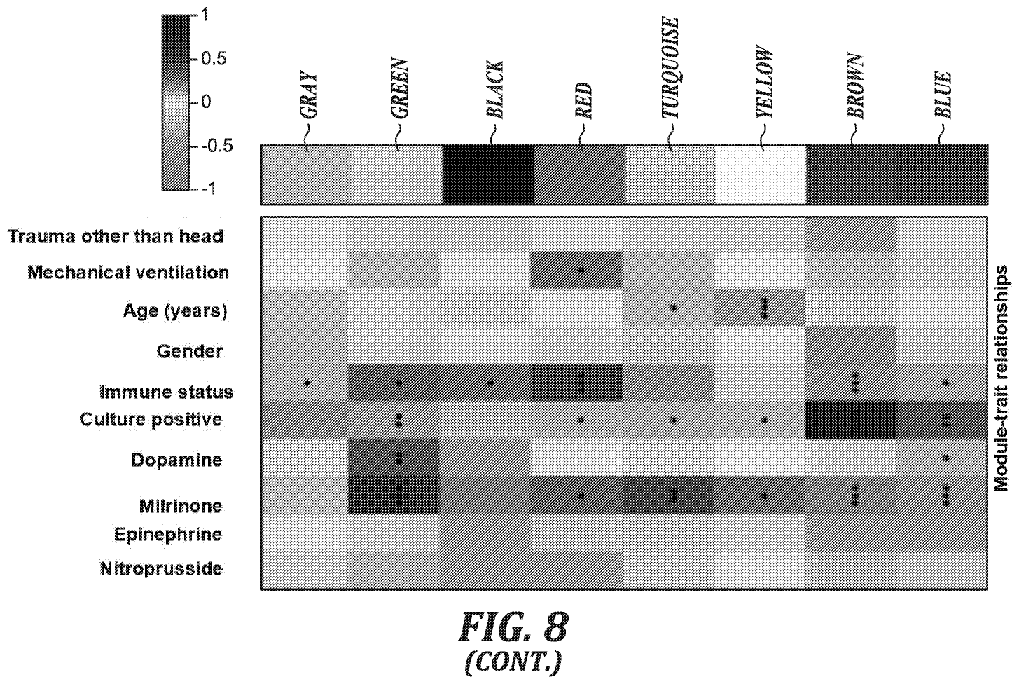

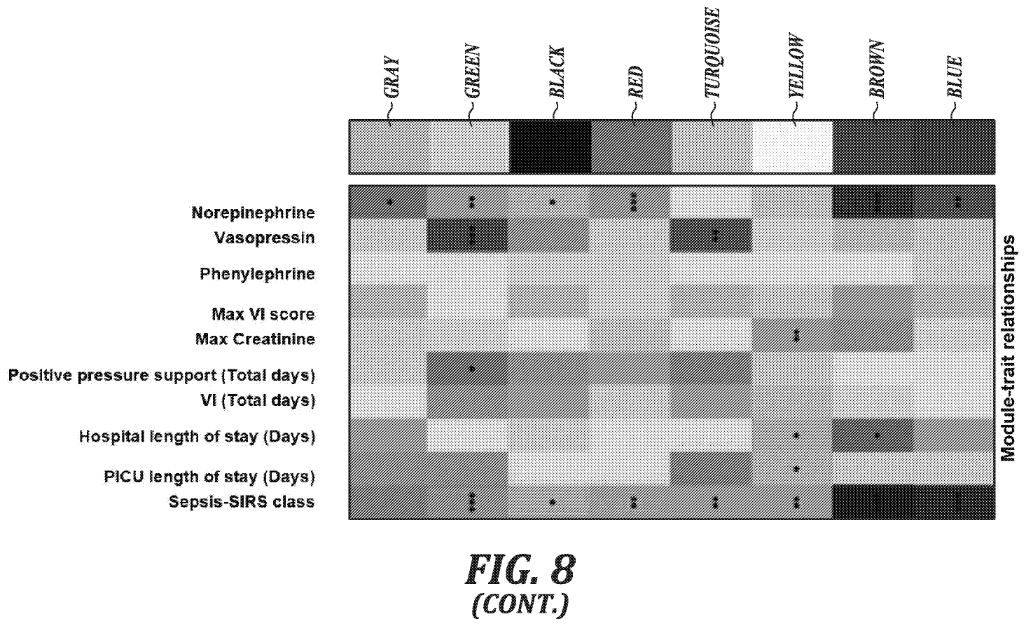

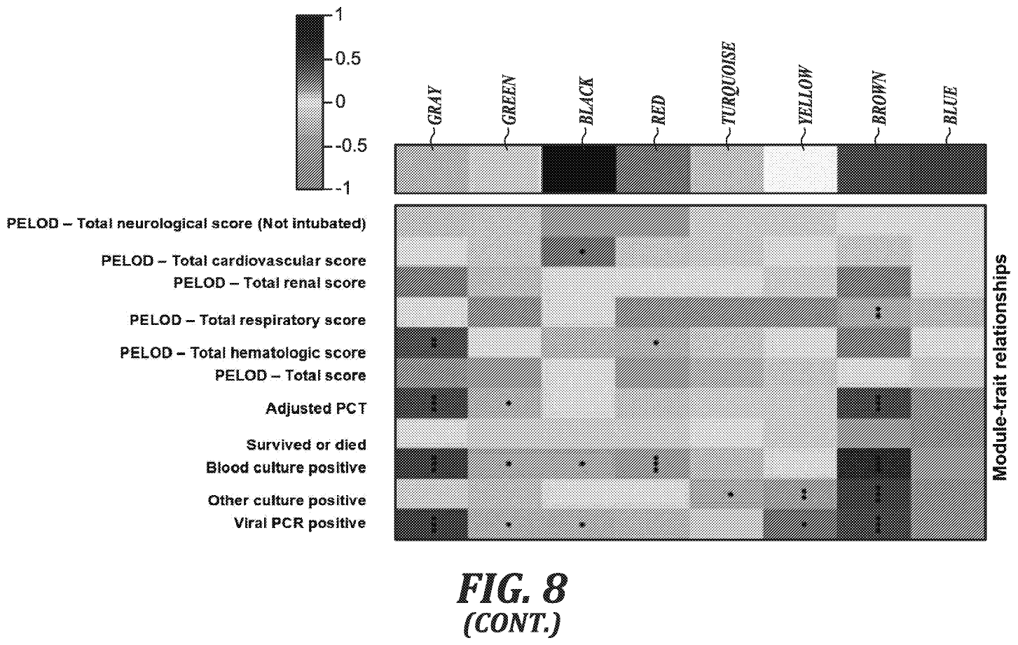

Seven co-expression modules, depicted with solid circles and squares in FIGS. 2A and 2B, were empirically found via WGCNA (cluster dendrogram shown in FIG. 7) with the associated traits described in FIGS. 2A and 2B with the complete module-trait relationship table shown in FIG. 8. Of these modules, the blue and brown WGCNA protein modules, which included 211 and 178 proteins, respectively, significantly correlated with expected clinical traits in sepsis patients, including SIRS criteria (increased heart and respiratory rates), positive bacterial cultures, and increased procalcitonin levels (FIG. 2A). Notably, the brown module included 27 proteins not previously reported as linked to sepsis (Table 3), and thus are potential sepsis biomarkers.

TABLE 3
“brown module” proteins not previously reported
to be involved in sepsis. Brown module refers to the
brown WGCNA module observed in FIGS. 2A and 2B.

| Gene name | Protein name | General protein type |
|---|---|---|
| ANK2 | Ankyrin-2 | Intracellular (adaptor protein) |
| BMP1 | Bone morphogenetic protein 1 | Protease |
| C1R | Complement Clr | Complement cascade |
| CA6 | Carbonic anhydrase 6 | Carbonic anhydrase |
| CCL16 | Chemokine (C-C motif) ligand 16 | Chemokine |
| CCL23 | Chemokine (C-C motif) ligand 23 | Chemokine |
| CD300C | Cluster of differentiation 300C | Receptor (binds |
| | | phosphatidylserine) |
| CRELD1 | Cysteine-rich with EGF-like | Intracellular |
| | domain protein 1 | (calcineurin/NFATc1 signaling) |
| CTSF | Cathepsin F | Protease |
| CTSV | Cathepsin V | Protease |
| EHMT2 | Euchromatic histone lysine | Intracellular (histone |
| | methyltransferase 2 | methyltransferase) |
| ERP29 | Endoplasmic reticulum protein 29 | Intracellular (ER protein) |
| FETUB | Fetuin B | Protease Inhibitor |
| FGF18 | Fibroblast growth factor 18 | Growth factor |
| FGF20 | Fibroblast growth factor 20 | Growth factor |
| FSTL3 | Follistatin-related protein 3 | Adhesion protein |
| GFRA1 | Glial cell derived neurotrophic factor | Receptor (binds GDNF and |
| | | NTN) |
| KYNU | Kynureninase | Tryptophan metabolite enzyme |
| MCL1 | Myeloid cell leukemia sequence 1 | Intracellular (anti-apoptotic |
| | (BCL2-Related) | protein) |
| MED1 | Mediator Complex Subunit 1 | Intracellular (gene transcription |
| | | protein) |
| MMP10 | Stromelysin-2 | Protease |
| NTF3 | Neurotrophin 3 | Growth factor |
| PIAS4 | Protein inhibitor of activated STAT 4 | Intracellular (sumoylation |
| | | mediator) |
| PRSS2 | Serine protease 2 (Anionic trypsinogen) | Protease |
| PTN | Pleiotrophin | Growth factor |
| TPSG1 | Tryptase gamma 1 | Protease |
| WIF1 | WNT inhibitory factor 1 | WNT pathway inhibitor |

Importantly, 6 blue WGCNA module proteins and 76 brown WGCNA module proteins were differentially expressed between the sepsis and INSI patients (Table 8). This demonstrates the invention can identify both known and novel proteins changed during sepsis but not during cardiopulmonary bypass-induced INSI.

In contrast to the blue and brown WGCNA modules, the green WGCNA module significantly correlated with expected cardiopulmonary bypass clinical traits, including changes in blood pressure encountered during the surgical procedure (shown in FIG. 2B as negative correlation with systolic and diastolic blood pressure [high values]), and milrinone usage, a medication for the prevention of low cardiac output syndrome after pediatric cardiac surgery (shown in FIG. 2B as positive correlation with milrinone usage) as well as an absence of infection (shown in FIG. 2A as negative correlation with culture positivity). Twelve proteins in the green WGCNA module were differentially expressed between the sepsis and cardiopulmonary bypass INSI patients (Table 8). Importantly, the clinical traits which correlate with the blue and brown WGCNA modules do not highly correlate with the proteins included in the green WGCNA module, indicating this analysis can assist in the identification of proteins that associate with clinical sepsis traits, but not with cardiopulmonary bypass-induced INSI traits.

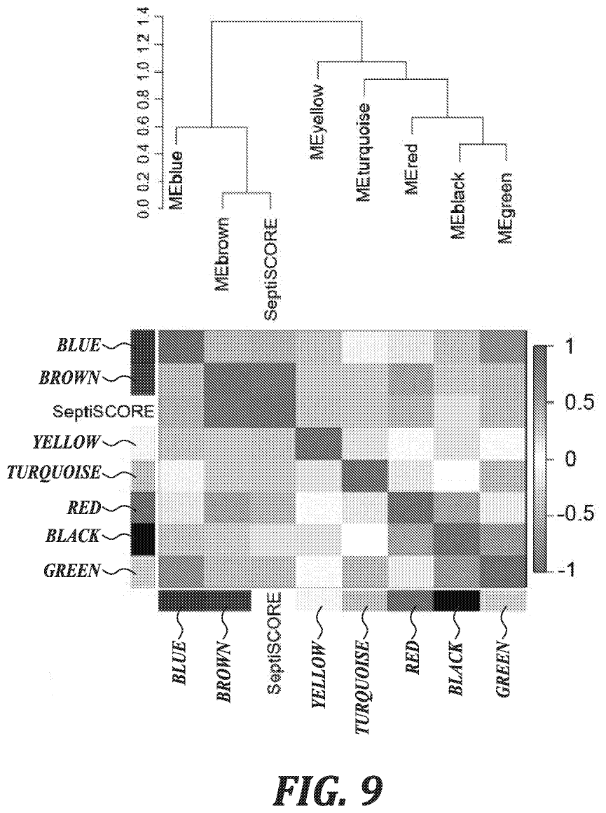

To further validate that the blue and brown modules include proteins that change specifically during sepsis, while the green module contains proteins that change during CPB INSI, correlations between these modules and patient SeptiSCORE values was examined. As depicted in the module eigengene correlation heatmap in FIG. 9, the blue and brown modules clustered together with the patient SeptiSCORE values and positively correlated with this trait parameter (r=0.41, p=0.001 and r=0.88, p<0.0001, respectively). In contrast, the yellow, turquoise, red, and green modules negatively correlated with the SeptiSCORE values of the patients (r=−0.39, p=0.002; r=−0.34, p=0.007; r=−0.45, p<0.0001; and r=−0.42, P=0.0007, respectively). The black and grey modules did not significantly correlate with SeptiSCORE changes. Overall, these correlations provide additional evidence that the blue and brown modules include proteins that change specifically during sepsis.

Identification of Potential Mechanisms of Disease

Two approaches to explore potential pathophysiological mechanisms of disease based on module proteins associated with clinical traits of sepsis (brown) and pediatric cardiac bypass procedures (green) differentially expressed between the sepsis and CPB INSI patients (Table 8) were utilized. The blue module proteins were not included in this analysis because only six proteins in this module were found to be differentially expressed between the sepsis and INSI patients (Table 8), and because the blue module proteins were not as strongly associated with sepsis as the brown module proteins.

First, the gene ontology analysis revealed that the brown module proteins associated with well-known sepsis pathways, such as acute phase, immunity, coagulation/fibrinolysis, and defense against bacteria responses (Table S6). In contrast, the green module proteins associated with hydrogen peroxide catabolism and response to reactive oxygen species (Table S6).

Although gene ontology analysis can assist identification of groups of proteins which associate with known sepsis responses, it does not determine how the interactions between these responses fit into an overall mechanism that leads to sepsis. Accordingly, Ingenuity Pathway Analysis (IPA) was used to identify specific protein-protein interaction networks to bridge these responses to each other.

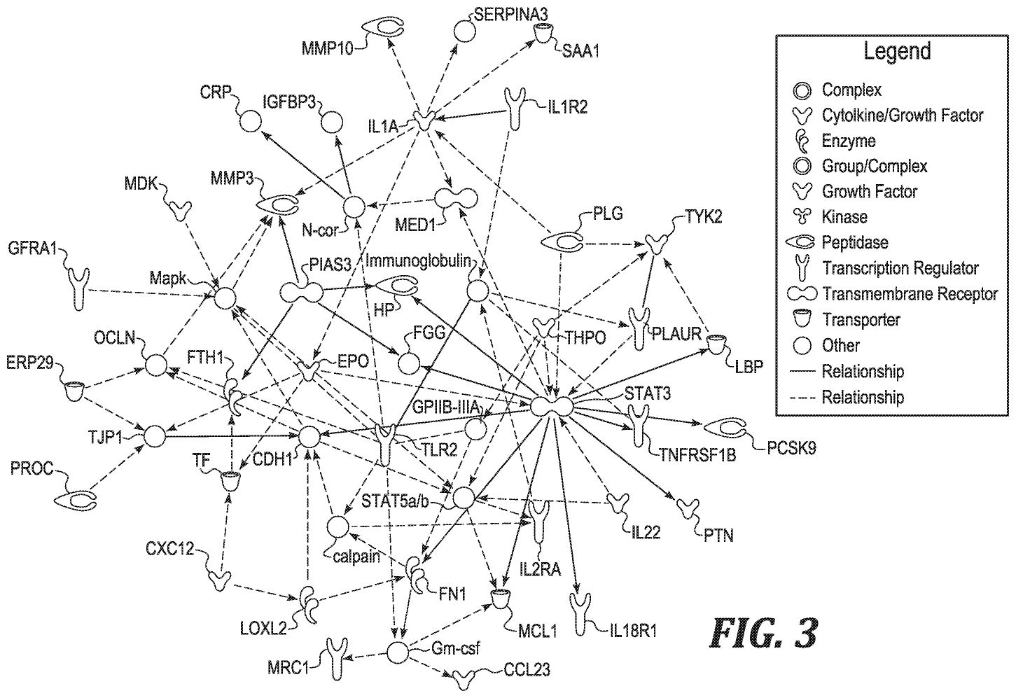

FIG. 3 shows that 33 of the 76 WGCNA brown module proteins were connected via direct or indirect interactions. The protein with the most relationships identified by IPA was signal transducer and activator of transcription-3 (STAT3), a key transcription factor for myeloid cell development (sepsis infantry leukocytes). Sixteen proteins that interact with STAT3 were identified. Five of these indirectly alter STAT3 phosphorylation and 11 exhibit expression directly or indirectly altered by STAT3 phosphorylation. All 16 of these proteins have known associations with pathophysiological mechanisms of sepsis (Table 6), including LPS-binding protein, haptoglobin, and fibrinogen gamma chain. Additionally, two of the proteins identified by IPA with greater than 3 interactions, PIAS3 and LOXL2, were not previously identified as sepsis mediators (FIG. 3). These proteins have potential roles in sepsis outcomes by modulating inflammation through inhibiting STAT3 activation and inducing fibrosis, respectively.

FIG. 3 also depicts an additional 15 proteins not included in the module proteins that associate with sepsis clinical traits described in FIG. 2A, but were added by IPA to improve network connectivity (details on how these 276 proteins were added into the analysis by IPA are provided in the Supplemental Methods and Materials section, below). Nine of the 15 proteins targeted by the invention's aptamer-based proteomics approach revealed interleukin (IL)-22 significantly increased in sepsis compared with the cardiopulmonary bypass patients (1.5-fold increase; p=0.0002).

### Discussion

Recently the Surviving Sepsis Campaign Group identified the top research priorities for sepsis. Included in these research priorities were questions such as: Can targeted/personalized/precision medicine approaches determine which therapies will work for which patients at which times? Should rapid diagnostic tests be implemented in clinical practice? What information identifies organ dysfunction? What mechanisms underlie sepsis-induced cellular and subcellular dysfunction? How does sepsis alter bio-energetics and/or metabolism? Clinical proteomics is currently at an inflection point in terms of contributing answers to these questions. The latest definition of sepsis, Sepsis-3.0, is based on the concept of life-threatening organ dysfunction secondary to a dysregulated host response to infection. Sepsis-3.0 continues to emphasize clinical criteria for defining sepsis, but this approach is associated with gaps in both sensitivity and specificity with associated area under the receiver operating characteristic (AUROC) curves in the range of 0.65-0.75. Biomarkers, with input from proteomics, can describe the host response, contributing to an improved definition of sepsis while expediting clinical diagnosis and improving sepsis treatment. The disclosed method thus utilizes a proteomics approach involving SOMAmers to identify serum proteins differentially expressed between children with sepsis and children with cardiopulmonary bypass infection-negative systemic inflammation. This approach leverages advances in microfluidics and enzyme-linked immunosorbent technology to bring proteomic analyses into real time to facilitate clinical decision making.

In summary, 111 serum proteins (through 112 SOMAmers) differentially expressed between the pediatric sepsis and INSI post-cardiopulmonary bypass patients were identified (Table 6). The serum proteins previously described in sepsis patients further validated the aptamer proteomics approach. Additionally, changes in specific protein groups (brown and blue WGCNA modules) strongly associated with clinical sepsis traits were identified (FIGS. 2A and 2B and Table 8). Some of the proteins significantly differentially expressed between the sepsis and INSI subjects, not previously identified as sepsis indicators, were discovered by the invention and included in the brown and blue modules. These proteins fall into three main categories: 1) Proteases involved in inflammation and fibrinolysis processes, including bone morphogenetic protein 1 (BMP1), cathepsin F (CTSF), cathepsin V (CTSV), matrix metallopeptidase 10 (MMP10), serine protease 2 (PRSS2), and tryptase gamma 1 (TPSG1); 2) Intracellular proteins involved in processes that can lead to inflammation, such as apoptosis, endoplasmic reticulum (ER) stress and DNA damage repair, including ankyrin 2 (ANK2), cysteine rich with EGF like domains 1 (CRELD1), euchromatic histone lysine methyltransferase 2 (EHMT2), ER protein 29 (ERP29), BCL2 family apoptosis regulator (MCL1), mediator complex subunit 1 (MED1) and protein inhibitor of activated STAT4 (PIAS4); and 3) Growth factors that regulate inflammation in different contexts, including fibroblast growth factor 18 (FGF18), fibroblast growth factor 20 (FGF20), neurotrophin 3 (NTF3), and pleiotrophin (PTN). Understanding the extracellular roles of intracellular proteins detected in serum is crucial because they can act as alarmins or danger-associated molecular patterns that can trigger and perpetuate the inflammatory response during sepsis. An additional protein differentially expressed between sepsis and INSI subjects includes Wnt inhibitor factor 1 (WIF1), which is thought to prevent barrier disruption of endothelial cells and therefore potentially protect against bacterial translocation into the bloodstream during infection.

Using IPA, interaction among the proteins associated with clinical traits of sepsis (brown module proteins) was analyzed. This led to identification of biological pathways potentially implicated in the mechanisms of disease of the pediatric sepsis cohort subjects. The IPA analysis identified known protein-protein interactions between different classes of proteins (e.g., protease, acute phase, inflammatory, extracellular matrix, and transcription factor proteins), suggesting that the hub proteins (proteins with more than 3 interactions) might intersect these pathways during pediatric sepsis. One of the most prominent biological pathways from the IPA analysis involved the STAT3 signaling pathway. Four of the five proteins upstream of STAT3 phosphorylation and activation (THPO, PLAUR, IL-22, and EPO) were increased in the IPA analysis of the sepsis subject cohort. Serum STAT3 levels were measured in the SOMAscan assay and did not significantly differ between the sepsis and INSI subject groups. STAT3 is an intracellular transcription factor phosphorylated upon activation and STAT3 discovery from the IPA analysis can indicate intracellular STAT3 activation by upstream proteins.

### CONCLUSION

The 1,305 aptamer SOMAscan panel proteomic screening tool identified 111 proteins that were significantly differentially expressed between sepsis and INSI cardiopulmonary pediatric ICU patient day 1 serum samples. This comparison between sepsis and cardiopulmonary-induced INSI patients revealed plausibility each of these conditions was responsible for the up- or down-regulation in differential serum protein expression attributable to sepsis and not cardiopulmonary bypass-induced INST. The WGCNA module analysis identified the brown and, to a lesser extent, blue modules as sets of proteins which differentiate between sepsis and cardiopulmonary-induced INSI among tested subjects.

### Methods and Materials

Patient Cohort, Specimen Collection, and Processing

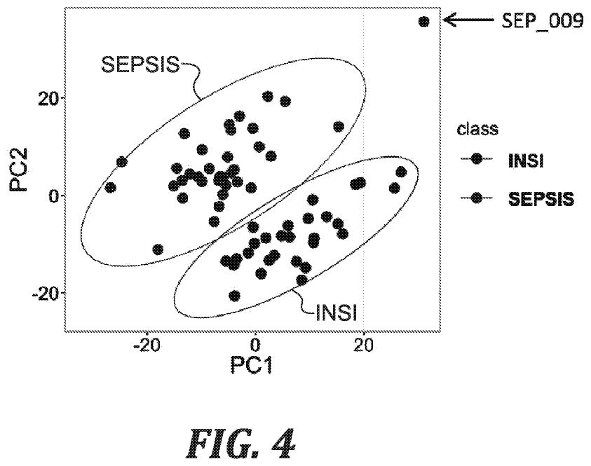

Forty clinical sepsis, and 30 post-cardio pulmonary bypass INSI patients, were recruited for the study (Zimmerman J J, et al. Diagnostic Accuracy of a Host Gene Expression Signature That Discriminates Clinical Severe Sepsis Syndrome and Infection-Negative Systemic Inflammation Among Critically Ill Children. Crit Care Med 2017; 45(4):e418-e425); however, not all patients yielded serum samples (FIG. 5). Therefore, samples from 35 sepsis and 28 cardiopulmonary bypass INSI patients were included in the proteomics assay. One sepsis patient's sample (SEP009) was identified as an outlier using principal components analysis and was excluded from downstream analysis, resulting in 34 patients in the sepsis group (FIG. 4). Further details are described in the Supplemental Methods and Materials section below.

Proteomics

Relative protein quantification was measured from patient serum samples with the SOMAscan platform by SOMAlogic (Boulder, Colo.) which consisted of 1,305 aptamers which had high affinity. Details of the SOMAscan process have been published (Gold L, et al. Aptamer-based multiplexed proteomic technology for biomarker discovery. PLoS One 2010; 5(12):e15004, Hathout Y, et al. Large-scale serum protein biomarker discovery in Duchenne muscular dystrophy. Proc Natl Acad Sci USA 2015; 112(23):7153-7158).

Bioinformatics Analyses

LIMMA (linear modeling) (Ritchie M E, et al. limma powers differential expression analyses for RNA-sequencing and microarray studies. Nucleic Acids Res 2015; 43(7):e47) and Boruta (random forests) (Kursa M B, Rudnicki W R. Feature Selection with the Boruta Package. J Stat Softw 2010; 36(11):1-13) analyses were used to identify differential protein expression between the sepsis and INSI groups. These methods identify significant differential expression changes in very different ways, thus increasing the probability of a protein being biologically or clinically meaningful. Specifically, proteins that were identified as being differentially expressed had a Benjamini-Hochberg adjusted p-value<0.01 following the LIMMA analysis and were not rejected by Boruta. This represented 8.6% ( 112/1305) of the total SOMAmer set.

Protein Module, Gene Ontology, Pathway, and Protein-Protein Interaction Analyses Weighted gene co-expression network analysis (WGCNA) was performed as described (Zhang B, Horvath S. A general framework for weighted gene co-expression network analysis. Stat Appl Genet Mol Biol 2005; 4: Article 17, Langfelder P, Horvath S. WGCNA: an R package for weighted correlation network analysis. BMC Bioinformatics 2008; 9:559) to identify protein modules that significantly correlated with clinical parameters and outcomes of the sepsis and INSI subjects. Correlations between these modules and patient SeptiSCORE values were also examined. SeptiSCORE derives from SeptiCyte™ LAB, a molecular test based on whole-blood expression levels of four genes (CEACAM4, LAMP1, PLA2G7 and PLACE) that is able to discriminate between systemic inflammatory response syndrome (SIRS)/INSI and sepsis (Zimmerman J J, et al. Diagnostic Accuracy of a Host Gene Expression Signature That Discriminates Clinical Severe Sepsis Syndrome and Infection-Negative Systemic Inflammation Among Critically Ill Children. Crit Care Med 2017; 45(4): e418-e425, McHugh L, et al. A Molecular Host Response Assay to Discriminate Between Sepsis and Infection-Negative Systemic Inflammation in Critically Ill Patients: Discovery and Validation in Independent Cohorts. PLoS Med 2015; 12(12): e1001916, Miller I i i R R, et al. Validation of a Host Response Assay, Septicyte LAB, for Discriminating Sepsis from SIRS in the ICU. Am J Respir Crit Care Med 2018). Gene ontology analysis was conducted with 153 the Database for Annotation, Visualization, and Integrated Discovery software 154 (DAVID), version 6.8, (david.ncifcrf.gov/summary sp). Ingenuity Pathway Analysis (IPA, qiagenbioinformatics.com/products/ingenuity-pathway-analysis/) was used to identify protein connectivity and experimentally validated protein-protein interactions. Further details are described in the Supplemental Methods and Materials section below.

### Supplemental Methods and Materials

Subject Cohort

The original subject cohort consisted of 40 children with clinically-overt sepsis, who had a confirmed or highly suspected infection (microbial culture orders, antimicrobial prescription); two or more systemic inflammation response syndrome criteria (SIRS, as defined in Levy M M, et al., 2001 SCCM/ESICM/ACCP/ATS/SIS International Sepsis Definitions Conference. Crit Care Med 2003, 31(4):1250-1256); and at least cardiovascular and/or pulmonary organ dysfunction. A second group of 30 children had undergone cardiopulmonary bypass for congenital heart surgery and were designated infection-negative systemic inflammation (INSI) controls (Zimmerman J J, et al., Diagnostic Accuracy of a Host Gene Expression Signature That Discriminates Clinical Severe Sepsis Syndrome and Infection-Negative Systemic Inflammation Among Critically Ill Children. Crit Care Med 2017, 45(4):e418-e425). Cardiopulmonary bypass is known to induce a SIRS response for ˜24 hours (Zimmerman J J, et al., Crit Care Med 2017, 45(4):e418-e425, supra). Of this subject cohort, 35/40 (87.5%) of the sepsis patients and 28/30 (28.3%) of the cardiopulmonary bypass patients yielded serum samples that could be used for proteomics analysis.

Specimen Collection and Processing

Serum samples were collected in serum separation tubes (Becton Dickinson) at day 1 of admission to the pediatric or cardiac intensive care unit (ICU). Post-centrifugation, samples were frozen at −70° C. to −80° C. They were thawed once, to remove a 150 μL aliquot for processing. The remaining sample and the 150 μL aliquot were refrozen, and the aliquot was shipped to SOMAlogic (Boulder, Colo.) for physical workup and analysis utilizing the SOMAmer methodology (Kraemer S, et al., From SOMAmer-based biomarker discovery to diagnostic and clinical applications: a SOMAmer-based, streamlined multiplex proteomic assay. PLoS One 2011, 6(10):e26332).

Proteomics

Relative protein quantification was measured from patient serum samples with the SOMAscan platform by SOMAlogic (Boulder, Colo.) that consisted of 1,305 aptamers which had high affinity. Serum samples were incubated with bead-coupled, fluorescently labelled SOMAmers, washed, and then the bead bound proteins were biotinylated. Subsequently, the biotinylated target protein-SOMAmer complexes were photocleaved from the beads, incubated with streptavidin beads, and washed further. Finally, the SOMAmers were eluted and quantified as representative of individual serum protein expression levels by hybridizing to SOMAmer-complementary oligonucleotide plate arrays. Standard samples were included on each plate to calibrate for inter-plate differences. The resulting raw intensities were then processed for hybridization and median signal normalization.

Bioinformatics Analysis

Pre-Processing: The SOMAlogic panel consists of 1,305 aptamers which had high affinity (SOMAmers). A total of 313 SOMAmers displayed a higher degree of correlation (Pearson correlation cut-off ≥0.8) and therefore redundancy of information content. The PCA was generated after removing highly correlated features (313/1305) that had an absolute pairwise correlation >=0.8 (function: findCorrelation, R package: caret, normal distribution ellipses: ggbiplot). One sepsis patient's sample, SEP009 was identified as an outlier by this method, and excluded from downstream analysis, leaving 34 subjects in the SEPSIS group after exclusion. The first two principal components accounted for approximately 24% of the variance in the data.

Differential protein expression analysis: LIMMA: The R package, LIMMA (Ritchie M E, et al., limma powers differential expression analyses for RNA-sequencing and microarray studies. Nucleic Acids Res 2015, 43(7):e47), designed to develop linear models from microarray data, was used to identify significant differences in protein expression levels between the sepsis and INSI groups. LIMMA fits a linear model to each row of data as represented by a SOMAmer. The columns represent individual patient samples belonging to either the sepsis or INSI group. For each SOMAmer the null hypothesis assumes that the coefficient vector would be equal to zero.

Differential protein expression analysis: Boruta: This R program is a wrapper for random forest classification (Kursa M B, Rudnicki W R. Feature Selection with the Boruta Package. J Stat Softw 2010, 36(11):1-13). “Shadow attributes” are created, which consist of random combinations of the original attributes. The shadow attributes, by virtue of their randomized origins, are expected to have low discriminatory power, with respect to separating the sepsis and INSI groups. Z-scores are computed when running random forest classification and the Z-scores of every “real” attribute are compared with the maximum Z score from the shadow attributes. A hit is recorded every time the Z-score of a real attribute is higher than the maximum Z score from the shadow attributes. Attributes with Z-score statistically significantly lower than the maximum Z-score from the shadow attributes are labeled as “rejected” and are removed at every iteration of the random forest classification. Attributes with a statistically significantly higher Z-score than the maximum Z-score from shadow attributes are labeled as “confirmed”. Some attributes that are not assigned importance within the pre-set number of iterations (99 by default, but could be a different quantity) are labeled as “tentative”. These tentative attributes are re-classified as confirmed or rejected by comparing the median Z score of attributes with the median Z-score of the best shadow attribute when using the ‘TentativeRoughFix’ method as implemented in the Boruta R package.

WGCNA: Weighted gene co-expression network analysis was performed as described (Zhang B, Horvath S. A general framework for weighted gene co-expression network analysis. Stat Appl Genet Mol Biol 2005, 4: Article 17; Langfelder P, Horvath S: WGCNA: an R package for weighted correlation network analysis. BMC Bioinformatics 2008, 9:559). Automatic network construction and module detection was performed using the R package WGCNA. A weighted protein correlation network was generated in which each of 1,305 nodes consisted of a SOMAmer with an expression value derived from the SOMAlogic assay. The edge connecting each pair of nodes represents the absolute value of the correlation of expression values of the corresponding SOMAmers. A co-expression similarity matrix containing this absolute value of correlation between every pair of SOMAmers is then converted into an adjacency matrix by raising the absolute value of correlation to a power ≥1. The soft-thresholding power is selected using the pickSoftThreshold algorithm from WGCNA. The probability that a node is connected with k other nodes in a biologically relevant real network has been shown to follow the power law p(k)˜k^{-γ} and to have a scale free topology (Zhang B, Horvath S. Stat Appl Genet Mol Biol 2005, 4: Article 17, supra).

A clustering dendrogram of SOMAmers with dissimilarity based on topological overlap was computed, and assigned specific module colors for easy reference. No dynamic tree-cutting algorithm was applied. The most significant clinical traits for each module were identified by binning with respect to p-value (high: p<0.001; moderate: 0.001<p≤0.01; low: 0.01<p≤0.05).

Gene ontology analysis: The Database for Annotation, Visualization, and Integrated Discovery software (DAVID), version 6.8, was utilized to determine the general functional annotations of the proteins contained in the different WGCNA modules that were shown to be differentially expressed between the sepsis and INSI subjects via LIMMA/Boruta analysis. The DAVID software determines a Benjamini-Hochberg P-value to determine gene ontology or molecular pathway enrichment. P-values<0.05 are considered strongly enriched in an annotation category.

Ingenuity pathway analysis (IPA): The significantly differentially expressed brown WGCNA module proteins (Table 6) were analyzed via IPA. Each protein was mapped to its corresponding object in Ingenuity's Knowledge Base. These molecules, called Network Eligible molecules, were overlaid onto a global molecular network developed from information contained in Ingenuity's Knowledge Base. Networks of Network Eligible Molecules were then algorithmically generated based on their connectivity. Additionally, the network was generated using the “Grow” feature present within IPA, which allows finding direct and indirect interactions between input molecules (WGCNA Brown module proteins) and adding 15 (user defined number) proteins that allow connecting more nodes within the input protein list. Regarding how the 15 “grow” proteins were added into the analysis, an incremental analysis was done by initially adding 10 proteins to help improve network connectivity between input proteins from the brown module of WGCNA. This resulted in IPA finding network interactions for 27 out of the 76 WGCNA brown module input proteins. The incremental addition of 15 proteins resulted in IPA finding network interactions for 33 out of the 76 WGCNA input brown module proteins. Since the addition of 5 proteins (to previous 10) by IPA did not significantly increase the network connectivity between the input proteins, the analysis was terminated at the addition of 15 proteins. The 5 additional proteins added by IPA to this analysis in addition to the initial 10 were: LOXL2 (Lysyl oxidase like 2), MAPK (Mitogen-activated protein kinases), STAT3 (Signal transducer and activator of transcription 3), STAT5a/b (Signal transducer and activator of transcription 5A), GPIIB-IIIA (Glycoprotein IIB-IIIA).

Other statistical analysis: For the patient characteristics evaluated in Table 4, continuous values were evaluated with the Mann-Whitney U test and categorical values were evaluated with the Fisher's exact test to determine p-values.

### Additional Tables

TABLE 4
patient population.

| | Sepsis (n = 34) | | INSI (n = 28) | | P-value |
|---|---|---|---|---|---|
| Age (range) | 9.2 | (0.1-17.5) | 7.5 | (0.76-16.6) | 0.4331 |
| Female (n, %) | 17 | (50) | 11 | (39.3) | 0.4497 |
| Culture results (n, % positive) | 25 | (73.5) | 1 | (3.6)* | <0.0001 |
| Pediatric Risk of Mortality, version III | 8.6 ± 6.6 | | 6.75 ± 4.5 | | 0.3831 |
| (admission score) | | | | | |
| Pediatric Logistic Organ Dysfunction | 4.6 ± 2.6 | | 5.0 ± 2.2 | | 0.3479 |
| score (day 1 score) | | | | | |
| Pediatric intensive care unit stay | 6.0 ± 6.2 | | 3.9 ± 7.12 | | 0.0003 |
| (duration, days) | | | | | |
| Hospital stay (duration, days) | 20.3 ± 20.0 | | 8.1 ± 7.85 | | <0.0001 |
| Cancer diagnosis (n, %) | 10.0 | (29.4) | 0.0 | (0) | 0.0013 |
| Immune status = Immune compromised | 13.0 | (38.2) | 1.0 | (3.8) | 0.0016 |
| (n, %) | | | | | |
| Mortality (n, %) | 1.0 | (2.9) | 0.0 | (0) | >0.9999 |
| SIRS INDICATORS: | | | | | |
| Systolic blood pressure (highest value) | 118.9 ± 16.6 | | 115.3 ± 19.0 | | 0.5761 |
| Systolic blood pressure (lowest value) | 78.4 ± 14.5 | | 75.1 ± 11.9 | | 0.2890 |
| Heart rate (highest value) | 158.4 ± 30.7 | | 126.5 ± 24.3 | | <0.0001 |
| Heart rate (lowest value) | 107.0 ± 25.8 | | 91.2 ± 19.3 | | 0.0111 |
| Respiratory rate (highest value) | 43.2 ± 14.7 | | 29.0 ± 6.5 | | <0.0001 |
| Respiratory rate (lowest value) | 20.9 ± 7.2 | | 15.5 ± 3.8 | | 0.0023 |
| Temperature (highest value) | 38.5 ± 1.2 | | 37.9 ± 0.5 | | 0.0203 |
| Temperature (lowest value) | 36.7 ± 0.9 | | 36.6 ± 0.7 | | 0.3235 |
| ACIDOSIS INDICATORS: | | | | | |

| | | | |
|---|---|---|---|
| Bicarbonate (highest value) | 20.1 ± 4.4 | 25.0 ± 2.1 | <0.0001 |
| Bicarbonate (lowest value) | 17.3 ± 4.1 | 23.1 ± 2.2 | <0.0001 |
| PCO2 (highest value) | 41.7 ± 9.8 | 50.7 ± 7.6 | 0.0008 |
| PCO2 (lowest value) | 34.5 ± 9.8 | 37.5 ± 6.2 | 0.1292 |

*One cardiopulmonary bypass-induced INSI patient tested positive for methicillin-resistant Staphylococcus aureus (MRSA) at PICU admission (as an aspect of routine MRSA surveillance screening) but did not display signs or symptoms of sepsis.
Continuous values are shown as mean ± standard deviation, and the Mann-Whitney U test was used to determine p-values for these variables. The Fisher's exact test was used to determine p-values for categorical variables. Note that there were some missing values for the acidosis indicators.
Bicarbonate high (sepsis n = 30, INSI n = 27); bicarbonate low (sepsis n = 16, INSI n = 15); PCO₂ high (sepsis n = 29, INSI n = 28); PCO2 low (sepsis n = 25, INSI n = 28).

Table 5 is a table of total differentially expressed proteins. INSI: Infection-negative systemic inflammation; LIMMA: Linear models for microarray data.

| | | | | | Log2 |
|---|---|---|---|---|---|
| | | Entrez | | | fold |
| | | Gene | Protein Name | Adjusted | (Sepsis |
| No. | SOMAmer | Symbol | (SOMAmer target) | P-Value* | vs. INSI |
| 1 | SL014896 | ANK2 | Ankyrin-2 | 6.84E−47 | −5.89 |
| 2 | SL001761 | TNNI3 | Troponin I, cardiac muscle | 2.24E−25 | −5.21 |
| 3 | SL004146 | IL1RL1 | Interleukin-1 receptor-like 1 | 1.83E−19 | 3.54 |
| 4 | SL003309 | LBP | Lipopolysaccharid e- | 1.98E−19 | 2.13 |
| | | | binding protein | | |
| 5 | SL000437 | HP | Haptoglobin | 2.25E−16 | 6.71 |
| 6 | SL000836 | HBA1 | Hemoglobin | 1.15E−15 | −6.18 |
| | | HBB | | | |
| 7 | SL003770 | SFRP1 | Secreted frizzled-related | 1.16E−15 | −3.94 |
| | | | protein 1 | | |
| 8 | SL000055 | CDH1 | Cadherin-1 | 1.47E−15 | −1.34 |
| 9 | SL007631 | SOST | Sclerostin | 2.05E−15 | −1.09 |
| 10 | SL006523 | MFGE8 | Lactadherin | 2.35E−14 | 2.83 |
| 11 | SL008381 | CTSF | Cathepsin F | 5.91E−14 | −0.93 |
| 12 | SL000572 | SAA1 | Serum amyloid A-1 protein | 3.49E−13 | 3.94 |
| 13 | SL008023 | HAPLN1 | Hyaluronan and | 3.94E−13 | −1.61 |
| | | | proteoglycan link protein 1 | | |
| 14 | SL004652 | WIF1 | Wnt inhibitory factor 1 | 1.09E−12 | −0.85 |
| 15 | SL010328 | MED1 | Mediator of RNA | 1.28E−12 | −1.21 |
| | | | polymerase II transcription | | |
| | | | subunit 1 | | |
| 16 | SL004152 | IL18R1 | Interleukin-18 receptor 1 | 1.51E−12 | 1.05 |
| 17 | SL004336 | FGF18 | Fibroblast growth factor 18 | 1.51E−12 | −1.63 |
| 18 | SL000462 | IGFBP1 | Insulin-like growth factor- | 1.51E−12 | −2.42 |
| | | | binding protein 1 | | |
| 19 | SL002704 | PTN | Pleiotrophin | 1.77E−12 | −3.95 |
| 20 | SL002508 | IL18BP | Interleukin-18- binding | 1.86E−12 | 2.46 |
| | | | protein | | |
| 21 | SL003994 | BMP1 | Bone morphogenetic | 3.32E−12 | −0.94 |
| | | | protein 1 | | |
| 22 | SL008102 | MDH1 | Malate dehydrogenase, | 6.73E−12 | −1.65 |
| | | | cytoplasmic | | |
| 23 | SL000051 | CRP | C-reactive protein | 1.30E−11 | 2.57 |
| 24 | SL019096 | PIAS4 | E3 SUMO-protein ligase | 1.50E−11 | 0.88 |
| | | | PIAS4 | | |
| 25 | SL000342 | CAT | Catalase | 4.35E−11 | −1.22 |
| 26 | SL002785 | NPPB | N-terminal pro-BNP | 6.43E−11 | 2.96 |
| 27 | SL004712 | CXCL12 | Stromal cell-derived factor 1 | 9.67E−11 | −1.48 |
| 28 | SL000420 | FTH1 FTL | Ferritin | 1.29E−10 | 3.06 |
| 29 | SL000508 | LTA LTB | Lymphotoxin alpha2:beta1 | 1.33E−10 | −0.75 |
| 30 | SL004858 | GFRA1 | GDNF family receptor | 1.90E−10 | −1.55 |
| | | | alpha-1 | | |
| 31 | SL003648 | GDI2 | Rab GDPdissociation | 2.72E−10 | −1.07 |
| | | | inhibitor beta | | |
| 32 | SL000598 | THPO | Thrombopoietin | 3.74E−10 | 2.32 |
| 33 | SL003302 | CCL23 | C-C motif chemokine 23 | 4.51E−10 | 1.65 |
| 34 | SL005694 | PRDX6 | Peroxiredoxin-6 | 4.72E−10 | −1.00 |
| 35 | SL003655 | TKT | Transketolase | 4.72E−10 | −1.09 |
| 36 | SL006910 | CTSV | Cathepsin L2 | 4.73E−10 | −1.51 |
| 37 | SL012774 | CRELD1 | Cysteine-rich with EGF- | 6.89E−10 | 1.28 |
| | | | like domain protein 1 | | |
| 38 | SL000524 | MMP3 | Stromelysin-1 | 7.02E−10 | 1.69 |
| 39 | SL004812 | TPI1 | Triosephosphate isomerase | 1.66E−09 | −1.26 |
| 40 | SL004921 | NME2 | Nucleoside diphosphate | 1.66E−09 | −1.14 |
| | | | kinase B | | |
| 41 | SL002525 | C2 | Complement C2 | 1.74E−09 | 0.65 |
| 42 | SL006542 | FCN2 | Ficolin-2 | 2.68E−09 | 0.60 |
| 43 | SL000248 | SERPIN | Alpha-1- antichymotrypsin | 2.93E−09 | 0.55 |
| | | A3 | | | |
| 44 | SL003542 | EHMT2 | Histone-lysine N- | 4.18E−09 | −0.69 |
| | | | methyltransferase EHMT2 | | |
| 45 | SL009324 | FSTL3 | Follistatin-related protein 3 | 5.56E−09 | 1.51 |
| 46 | SL003301 | CCL23 | Ck-beta-8-1 | 5.70E−09 | 1.29 |
| 47 | SL004876 | SERPIN | Kallistatin | 6.53E−09 | −0.95 |
| | | A4 | | | |
| 48 | SL000545 | KLKB1 | Plasma kallikrein | 9.20E−09 | −0.84 |
| 49 | SL000145 | IL1R2 | Interleukin-1 receptor type 2 | 1.29E−08 | 1.35 |
| 50 | SL002621 | MDK | Midkine | 1.59E−08 | −2.65 |
| 51 | SL014069 | PCSK7 | Proprotein convertase | 2.81E−08 | 0.90 |
| | | | subtilisin/kexin type 7 | | |
| 52 | SL000048 | PROC | Vitamin K-dependent | 2.82E−08 | −0.60 |
| | | | protein C | | |
| 53 | SL004579 | MRC1 | Macrophage mannose | 3.07E−08 | 1.10 |
| | | | receptor 1 | | |
| 54 | SL017289 | UBB | PolyUbiquitin K48-linked | 3.17E−08 | −0.97 |
| 55 | SL004060 | ECE1 | Endothelin- converting | 3.18E−08 | −0.69 |
| | | | enzyme 1 | | |
| 56 | SL010524 | WNK3 | Serine/threonine- protein | 3.51E−08 | −0.86 |
| | | | kinase WNK3 | | |
| 57 | SL000455 | JUN | Transcription factor AP-1 | 4.50E−08 | 0.64 |
| 58 | SL004145 | TNFRSF | Tumor necrosis factor | 5.02E−08 | 0.46 |
| | | 14 | receptor superfamily | | |
| | | | member 14 | | |
| 59 | SL000254 | ALB | Serum albumin | 5.03E−08 | −1.13 |
| 60 | SL003522 | ERP29 | Endoplasmic reticulum | 5.14E−08 | −0.85 |
| | | | resident protein 29 | | |
| 61 | SL003341 | FGG | Fibrinogen gamma chain | 6.29E−08 | 1.52 |
| 62 | SL004492 | TLR2 | Toll-like receptor 2 | 6.48E−08 | 0.70 |
| 63 | SL011510 | SST | Somatostatin-28 | 6.96E−08 | 0.61 |
| 64 | SL001800 | TNFRSF | Tumor necrosis factor | 6.96E−08 | 1.03 |
| | | 1B | receptor superfamily | | |
| | | | member 1B | | |
| 65 | SL012707 | PCSK9 | Proprotein convertase | 7.28E−08 | 0.92 |
| | | | subtilisin/kexin type 9 | | |
| 66 | SL002506 | PLAUR | Urokinase plasminogen | 7.49E−08 | 0.75 |
| | | | activator surface receptor | | |
| 67 | SL004739 | ITIH4 | Inter-alpha-trypsin inhibitor | 7.49E−08 | 0.78 |
| | | | heavy chain H4 | | |
| 68 | SL004489 | TLR4 | Toll-like receptor 4 | 7.77E−08 | 1.31 |
| 69 | SL004742 | AFM | Afamin | 7.77E−08 | −0.69 |
| 70 | SL008178 | DPT | Dermatopontin | 7.81E−08 | −0.89 |
| 71 | SL000541 | PLG | Plasminogen | 2.97E−07 | −0.82 |
| 72 | SL010288 | CA6 | Carbonic anhydrase 6 | 3.01E−07 | −2.42 |
| 73 | SL004852 | CD274 | Programmed cell death 1 | 3.04E−07 | 1.05 |
| | | | ligand 1 | | |
| 74 | SL002528 | PLA2G2 A | Phospholipase A2, | 3.62E−07 | 3.07 |
| | | | membrane associated | | |
| 75 | SL004326 | TNFSF1 8 | Tumor necrosis factor | 5.93E−07 | 0.39 |
| | | | ligand superfamily member 18 | | |
| 76 | SL000426 | FN1 | Fibronectin | 6.40E−07 | −1.26 |
| 77 | SL003755 | MCL1 | Induced myeloid leukemia | 8.54E−07 | 0.70 |
| | | | cell differentiation protein | | |
| | | | Mcl-1 | | |
| 78 | SL003300 | CCL16 | C-C motifchemokine 16 | 9.47E−07 | −1.76 |
| 79 | SL000022 | FGA FGB | D-dimer | 9.62E−07 | 1.50 |
| | | FGG | | | |
| 80 | SL004821 | S100A4 | Protein S100-A4 | 9.62E−07 | −0.93 |
| 81 | SL000497 | LAMA1 | Laminin | 9.89E−07 | 0.92 |
| | | LAMB1 | | | |
| | | LAMC1 | | | |
| 82 | SL000321 | C5 C6 | Complement C5b-C6 | 1.11E−06 | 0.37 |
| | | | complex | | |
| 83 | SL004642 | ADAM9 | Disintegrin and | 1.72E−06 | 0.94 |
| | | | metalloproteinase domain- | | |
| | | | containing protein 9 | | |
| 84 | SL006777 | FETUB | Fetuin-B | 1.76E−06 | −1.13 |
| 85 | SL007306 | FAM3B | Protein FAM3B | 1.90E−06 | −0.94 |
| 86 | SL004097 | SMAD3 | Mothers against | 2.17E−06 | 0.71 |
| | | | decapentaplegic homolog 3 | | |
| 87 | SL013969 | KYNU | Kynureninase | 2.54E−06 | 1.03 |
| 88 | SL000382 | CKB CKM | Creatine kinase M- | 3.55E−06 | −2.65 |
| | | | type: Creatine kinase B-type | | |
| | | | heterodimer | | |
| 89 | SL004919 | PRDX1 | Peroxiredoxin-1 | 3.71E−06 | −0.86 |
| 90 | SL004260 | RETN | Resistin | 5.72E−06 | 1.15 |
| 91 | SL004338 | FGF20 | Fibroblast growth factor 20 | 6.65E−06 | −0.33 |
| 92 | SL007651 | FGF23 | Fibroblast growth factor 23 | 8.34E−06 | −1.33 |
| 93 | SL000045 | IGFBP3 | Insulin-like growth factor- | 1.18E−05 | −0.81 |
| | | | binding protein 3 | | |
| 94 | SL010388 | PRSS2 | Trypsin-2 | 1.46E−05 | 1.61 |
| 95 | SL008486 | LGALS9 | Galectin-9 | 1.88E−05 | 0.63 |
| 96 | SL000310 | C1R | Complement Clr | 1.99E−05 | 1.05 |
| | | | subcomponent | | |
| 97 | SL014270 | CD300C | CMRF35-like molecule 6 | 2.16E−05 | 0.75 |
| 98 | SL000645 | MMP10 | Stromelysin-2 | 3.64E−05 | 1.23 |
| 99 | SL010489 | CAMK1 | Calcium/calmoduli n- | 3.97E−05 | 0.36 |
| | | | dependent protein kinase | | |
| | | | type 1 | | |
| 100 | SL000408 | EPO | Erythropoietin | 5.81E−05 | 1.53 |
| 101 | SL007108 | IRF1 | Interferon regulatory | 7.20E−05 | 0.21 |
| | | | factor 1 | | |
| 102 | SL010619 | TPSG1 | Tryptase gamma | 7.76E−05 | −0.47 |
| 103 | SL003189 | CCL19 | C-C motifchemokine 19 | 0.000102049 | 1.37 |
| 104 | SL003305 | IL2RA | Interleukin-2 receptor | 0.000153499 | 0.63 |
| | | | subunit alpha | | |
| 105 | SL002722 | CD38 | ADP-ribosyl cyclase/cyclic | 0.000190617 | 0.21 |
| | | | ADP-ribose hydrolase 1 | | |
| 106 | SL004347 | IL22 | Interleukin-22 | 0.000245517 | 0.59 |
| 107 | SL000383 | CKM | Creatine kinase M-type | 0.000433221 | −1.52 |
| 108 | SL004359 | NTF3 | Neurotrophin-3 | 0.000636868 | −0.49 |
| 109 | SL008931 | CD177 | CD177 antigen | 0.000766 | 1.31 |
| 110 | SL000325 | C9 | Complement component C9 | 0.001072455 | 0.60 |
| 111 | SL006705 | PFDN5 | Prefoldin subunit 5 | 0.001304314 | −0.60 |
| 112 | SL012822 | PRSS22 | Brain-specific serine | 0.005810436 | 0.61 |
| | | | protease 4 | | |

*Benjamini-Hochberg multiple testing correction from LIMMA

TABLE 6
proteins identified in the SOMAscan screen with
established prior associations to sepsis.

| Entrez Gene | | References |
|---|---|---|
| Symbol | Protein Name | (PMID/PMCID) |
| ADAM9 | Disintegrin and metalloproteinase | 27990250 |
| | domain-containing protein 9 | |
| AFM | Afamin | 23981841 |
| ALB | Serum Albumin | 26158725, |
| | | 20149587, |
| | | 22801198 |
| APOE | Apolipoprotein E (isoform E4) | 24266763, |
| | | 24655576, |
| | | 19157344 |
| CAMK1 | Calcium/Calmodulin Dependent | 21372190, |
| | Protein Kinase I | 23091438 |
| CAT | Catalase | 29484685, |
| | | 28167245, |
| | | 25999034 |
| CD177 | NB1 glycoprotein | 26829180, |
| | | 27568821 |
| CD274 | Programmed cell death 1 ligand 1 | 27864994 |
| CDH1 | E-Cadherin | 8688260 |
| CRP | C-reactive protein | 26150837 |
| CXCL12 | Stromal-derived-factor 1 | 28562124, |
| | | 27832827 |
| ECE1 | Endothelin-converting enzyme-1 | 15733912 |
| EPO | Erythropoietin | 9003476, |
| | | 15469576, |
| | | 16235474 |
| FCN2 | Ficolin-2 | 28407349, |
| | | 24227370 |
| FGA FGB | D-dimer | 26586287, |
| FGG | | 24030119, |
| | | 23497204 |
| FGF23 | Fibroblast growth factor 23 | 26728476 |
| FGG | Fibrinogen gamma chain | 26277871, |
| | | 27512924 |
| FN1 | Fibronectin | 8445454, |
| | | 22837119 |
| FTH1 FTL | Ferritin | 28126563, |
| | | 18001337 |
| HBA1 HBB | Hemoglobin | PMC3642527, |
| | | 27737630 |
| HP | Haptoglobin | 26239984, |
| | | 23372690 |
| IGFBP1 | Insulin-like growth factor- | 15009554, |
| | binding protein 1 | 12107211 |
| IGFBP3 | Insulin-like growth factor- | 23611528 |
| | binding protein 3 | |
| IL18BP | Interleukin-18-binding protein | 11497494 |
| IL18R1 | Interleukin-18 receptor 1 | 25538794 |
| IL1R2 | Interleukin-1 receptor type 2 | 27984536, |
| | | 24561564 |
| IL1RL1 | Interleukin-1 receptor-like 1 | 26354344, |
| | | 25850080 |
| IL22 | Interleukin-22 | 20220564 |
| IL2RA | Interleukin-2 receptor subunit | 24646167, |
| | alpha | 28155994, |
| | | 23531337 |
| ITIH4 | Inter-alpha-trypsin inhibitor | 19324226, |
| | heavy chain H4 | 20520583 |
| KLKB1 | Plasma kallikrein | 27046148, |
| | | 22442348, |
| | | 22352684 |
| LAMA1 LAMB1 | Laminin | 10583445 |
| LAMC1 | | |
| LBP | Lipopolysaccharide-binding | 24225281, |
| | protein | 24057110 |
| LTA | Lymphotoxin-alpha, -beta | 9050752, |
| | | 21366408 |
| LTB | | |
| MFGE8 | Lactadherin | 20882259 |
| MMP3 | Stromelysin-1 | 21439766 |
| MRC1 | Macrophage mannose receptor 1 | 29383956, |
| | | 25650730, |
| | | 24637679, |
| | | 24114918 |
| NPPB | N-terminal pro-BNP | 27002627, |
| | | 27380528 |
| PCSK9 | Proprotein convertase subtilisin/ | 26756586, |
| | kexin type 9 | 25320235 |
| PLA2G2A | Phospholipase A2, membrane | 22681048 |
| | associated | |
| PLAUR | Plasminogen activator, urokinase | 24882949, |
| | receptor | 25043869, |
| | | 26615223 |
| PLG | Angiostatin | 16368651 |
| PROC | Vitamin K-dependent protein C | 21737232 |
| RETN | Resistin | 28424824, |
| | | 25364554, |
| | | 25343379, |
| | | 23147079, |
| | | 22699030 |
| SAA1 | Serum amyloid A-1 protein | 12235722, |
| | | 23984377, |
| | | 28655573 |
| SERPINA3 | Alpha-1-antichymotrypsin | 24266763 |
| SERPINA4 | Kallistatin | 28542440, |
| | | 25930108, |
| | | 24467264 |
| SOST | Sclerostin | 27621111 |
| SST | Somatostatin-28 | 20307604, |
| | | 24457113 |
| THPO | Thrombopoietin | 24887960, |
| | | 22746320 |
| TLR2 | Toll-like receptor 2 | PMC4240815 |
| TLR4 | Toll-like receptor 4 | PMC4240815 |
| TNFRSF1 B | Tumor necrosis factor receptor | 15526005 |
| | superfamily member 1B | |
| TNFSF18 | Tumor necrosis factor ligand | 27124414 |
| | superfamily member 18 | |
| TNNI3 | Troponin I, Cardiac Muscle | 20149590, |
| | | 27077648 |

TABLE 7
tests for confounding variables. INSI: Infection-negative systemic inflammation.

| Stratification | Split | # | Significant Proteins (Boruta) |
|---|---|---|---|
| Sex | Sepsis Male (n = 17) vs. | 4 | GPC6, KLK3, CPB2, ALB* |
| | Sepsis Female (n = 17) | | |
| | INSI male (n = 17) | 4 | LCMT1, CD83, CA9, IL12A/IL12B |
| | vs. INSI female (n = 11) | | |
| Age | Sepsis < 11 years (n = 16) | 29 | MAP2K2, WFIKKN2, PGD, CMA1, |
| | vs. Sepsis ≥ 11 years | | *CXCL12, *HAPLN1, IL5RA, CTSD, |
| | (n = 18) | | NRXN1, UNC5D, RAC3, BCAN, |
| | | | SEMA3E, KLK4, RTN4R, FGF12, |
| | | | NCR3, SPP1, IBSP, LDHB, LCK, |
| | | | CNTN2, IL20, PDE9A, MMP16, |
| | | | NCAM1, STX1A, GPC3, TG |
| | INSI < 11 years (n = 19) | 12 | MMP13, SPOCK2, CTSD, F9, PIANP, |
| | vs. | | IGFBP5, RGMA, F3, SERPINF1, |
| | INSI ≥ 11 years (n = 9) | | KLK3, SET, SELL |
| Immune | Immunocompetent | 14 | TNFRSF17, FCER2, *FTH1/FTL, |
| Status | sepsis (n = 21) vs. | | IGHA1 IGHA2, CST6, JAG1, |
| | Immunocompromised | | *LGALS9, MIF, CFP, CCL14, NTRK3, |
| | sepsis (n = 13) | | MUC1, CD5L, IGM |
| | Immunocompetent INSI | | Analysis could not be completed due to |
| | (n = 27) vs. | | significantly unbalanced groups |
| | Immunocompromised | | |
| | INSI (n = 1) | | |
| Cancer** | Cancer sepsis (n = 10) vs. | 12 | TNFRSF17, FCER2, *FTH1/FTL, |
| | non-cancer sepsis (n = 24) | | IGHA1 IGHA2, JAG1, CFP, CD5L, |
| | | | GDF15, AFP, AURKB, DSC3, IGM |
| Viral co- | Sepsis and viral co- | 13 | NRCAM, SERPINA7, NTRK3, IBSP, |
| infection in | infection (n = 10) vs. | | GPC3, ROBO2, SPP1, NOTCH3, |
| sepsis | sepsis and no viral co- | | IFNB1, EPHB2, L1CAM, LEPR, GDF2 |
| patients | infection (n = 24) | | |

*Protein that is amongst the 111 differentially expressed proteins in Table 4.
**All cancer cases fell within the sepsis group, with none in the INSI group.

TABLE 8
differentially expressed proteins and WGCNA module designation.
LIMMA: Linear models for microarray data.

| WGCNA | |
|---|---|
| Module | LIMMA/Boruta differentially expressed proteins (Entrez gene IDs) |
| Turquoise | C2, C5/C6, C9, CCL19, CD274, CD38, LGALS9, PCSK7, SST, TLR TNFRSF14 |
| Blue | CKB/CKM, FAM3B, IRF1, JUN, SMAD3, TNFSF18 |
| Brown | ADAM9, AFM, ALB, ANK2, BMP1, C1R, CA6, CAMK1, CCL16, CCL |
| | (SOMAmers: 2913-1_2, 3028-36_2), CD177, CD300C, CRELD1, CRP, CTS |
| | CTSV, CXCL12, ECE1, EHMT2, EPO, ERP29, FCN2, FETUB, FGA/FGB/FG |
| | FGF18, FGF20, FGG, FN1, FSTL3, FTH1/FTL, GFRA1, HBA1/HBB, H IGFBP1, |
| | IGFBP3, IL18BP, IL18R1, IL22, IL1R2, IL1RL1, IL2RA, ITIH4, KLKB KYNU, |
| | LAMA1/LAMB1/LAMC1, LBP, LTA/LTB, MCL1, MDK, MED1, MFGE |
| | MMP10, MMP3, MRC1, NPPB, NTF3, PCSK9, PIAS4, PLA2G2A, PLAUR, PL |
| | PROC, PRSS2, PTN, SAA1, SERPINA4, SOST, THPO, TLR2, TNFRSF1 TNNI3, |
| | TPSG1, WIF1, CDH1, SERPINA3, SFRP1 |
| Yellow | DPT, HAPLN1, RETN |
| Green | CAT, FGF23, GDI2, MDH1, PFDN5, PRDX1, PRDX6, S100A4, TKT, TPI1, UB |
| | WNK3 |
| Red | None |
| Black | NME2 |
| Grey | CKM, PRSS22 |

TABLE 9
gene ontology analysis.

| Differentially | |
|---|---|
| expressed proteins | BP direct gene ontology terms for WGCNA module proteins |
| analyzed | (Benjamini p-value < 0.05) |
| Brown module | GO:0006953: Acute-phase response |
| proteins (76 proteins) | GO:0022617: Extracellular matrix disassembly |
| | GO:0002576: Platelet degranulation |
| | GO:0042730: Fibrinolysis |
| | GO:0007165: Signal transduction |
| | GO:0006955: Immune response |
| | GO:0070374: Positive regulation of ERK1 and ERK2 cascade |
| | GO:0031639: Plasminogen activation |
| | GO:0006508: Proteolysis |
| | GO:0007596: Blood coagulation |
| | GO:0030198: Extracellular matrix organization |
| | GO:0050830: Defense response to Gram-positive bacterium |
| | GO:0009267: Cellular response to starvation |
| | GO:0072378: Blood coagulation, fibrin clot formation |
| | GO:0007267: Cell-cell signaling |
| | GO:0044267: Cellular protein metabolic process |
| | GO:0050729: Positive regulation of inflammatory response |
| | GO:0006954: Inflammatory response |
| | GO:0090277: Positive regulation of peptide hormone secretion |
| | GO:0050918: Positive chemotaxis |
| | GO:0050714: Positive regulation of protein secretion |
| | GO:0008228: Opsonization |
| | GO:0070527: Platelet aggregation |
| | GO:0034116: Positive regulation of heterotypic cell-cell adhesion |
| Green module | GO:0042744: Hydrogen peroxide catabolic process |
| proteins (12 proteins) | GO:0000302: Response to reactive oxygen species |

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for detecting sepsis in a subject, comprising:
contacting a biological sample obtained from the subject with an affinity reagent that specifically binds to a biomarker selected from the biomarkers disclosed in Table 1, and
detecting differential expression of the biomarker by detecting binding of the affinity reagent to the biomarker, wherein differential expression of the biomarker indicates sepsis in the subject.

2. The method of claim 1, comprising contacting the biological sample with a plurality of affinity reagents that specifically bind to a plurality of biomarkers selected from the biomarkers disclosed in Table 1.

3. The method of claim 1 or claim 2, wherein the biomarker or plurality of biomarkers is selected from the biomarkers disclosed in Table 3.

4. The method of one of claims 1-3, wherein the method further comprises contacting the sample obtained with one or more affinity reagents that bind to one or more biomarkers selected from the biomarkers disclosed in Table 6.

5. The method of claim 1, wherein the biomarker is a protease.

6. The method of one of claims 1-5, wherein the biomarker is selected from BMP1, CTSF, CTSV, MMP10, PRSS2, and TPSG1, or the plurality of biomarkers comprises one or more of BMP1, CTSF, CTSV, MMP10, PRSS2, or TPSG1.

7. The method of claim 1, wherein the biomarker is an intracellular protein.

8. The method of one of claims 1-4 and 7, wherein the biomarker is selected from ANK2, CRELD1, EHMT2, ERP29, MCL1, MED1, and PIAS4, or the plurality of biomarkers comprises one or more of ANK2, CRELD1, EHMT2, ERP29, MCL1, MED1, and PIAS4.

9. The method of claim 1, wherein the biomarker is a growth factor.

10. The method of one of claims 1-4 and 9, wherein the biomarker is selected from FGF18, FGF20, NTF3, and PTN, or the plurality of biomarkers comprises one or more of FGF18, FGF20, NTF3, and PTN.

11. The method of claim 1, wherein the biomarker is WIF1.

12. The method of claim 4, wherein the one or more biomarkers selected from the biomarkers disclosed in Table 6 is selected from THPO, PLAUR, IL-22, and EPO.

13. The method of one of claims 1-12, wherein the biological sample is a blood, serum, or plasma.

14. The method of one of claims 1-13, further comprising obtaining the biological sample from the subject.

15. The method of one of claims 1-14, wherein the subject is human.

16. The method of claim 15, wherein the subject is less than about 20 years old.

17. The method of one of claims 1-16, wherein the affinity reagent is or comprises an antibody, an antibody fragment or derivative, or an aptamer.

18. The method of claim 17, wherein the affinity reagent is or comprises an aptamer that comprises an oligonucleotide, peptide, or protein.

19. The method of one of claims 1-18, wherein the affinity reagent is immobilized to a surface.

20. The method of one of claims 1-19, wherein the affinity reagent is immobilized to a bead.

21. The method of claim 20, wherein the affinity reagent is immobilized to the bead by a cleavable linker, wherein the affinity reagent is optionally an aptamer.

22. The method of one of claims 1-21, wherein the affinity reagent is detectable labeled.

23. The method of claim 22, wherein the detectable label comprises a fluorescence molecule.

24. The method of claim 23, wherein binding of the affinity reagent to the biomarker is detected by fluorescence.

25. The method of one of claims 1-24, wherein the aptamer is in an aptamer complex that comprises the aptamer linked to a bead with a cleavable linker, and a detectable label.

26. The method of one of claims 1-25, wherein detecting differential expression comprises comparing the binding level to a reference standard.

27. The method of claim 26, wherein the reference standard is obtained from one or more subjects with infection-negative systemic inflammation (INSI).

28. The method of claim 26, wherein the reference standard is obtained from one or more subjects without sepsis.

29. The method of claim 26, wherein differential expression is determined when the binding level significantly differs from the reference standard.

30. The method of one of claims 1-29, further comprising treating the subject that is determined to have sepsis.

31. The method of claim 30, wherein treating the subject comprises administering antibiotics, administering an intervention to control or maintain blood pressure, administering an intervention to control or maintain body temperature, or any combination thereof.

32. A method of monitoring sepsis in a subject, comprising performing the method recited in one of claims 1-31 at two or more time points.

33. The method of claim 32, wherein at least one of the two or more time points occurs during or after treatment of the subject for sepsis.

34. The method of claim 32, wherein a reduced detected differential expression of the biomarker indicates an amelioration of sepsis in the subject.

35. A method of monitoring the efficacy of treatment of sepsis in a subject, comprising performing the method of claim 33, wherein a reduction of detected differential expression of the biomarker over time after treatment indicates the efficacy of the treatment.

36. A kit, comprising an affinity reagent that specifically binds to a biomarker selected from the biomarkers disclosed in Table 1, and written indicia for performing the method of any one of claims 1-35.

37. The kit of claim 36, wherein the affinity reagent is an antibody, an antibody fragment or derivative, or an aptamer.

38. The kit of claim 36, wherein the affinity reagent is immobilized to a surface.

39. The kit of claim 36, wherein the affinity reagent is immobilized to a bead.

40. The kit of claim 39, wherein the affinity reagent is immobilized to the bead by a cleavable linker.

41. The kit of claim 36, wherein the affinity reagent is detectably labeled.

42. The kit of claim 37, wherein the aptamer is or comprises an oligonucleotide, peptide, or protein.

43. The kit of claim 36, comprising a plurality of different affinity reagents that specifically bind to two or more biomarkers selected from the biomarkers disclosed in Table 5.
